(19) United States
(12) Patent Application Publication
(10) Pub. No.: US 2024/0025867 A1
(43) Pub. Date: Jan. 25, 2024

(54) CATALYSTS FOR BENZOXAZINE

(71) Applicant: LUXEMBOURG INSTITUTE OF SCIENCE AND TECHNOLOGY(LIST), Esch-sur-Alzette (LU)

(72) Inventors: Pierre VERGE, Esch-sur-Alzette (LU); Antoine ADJAOUD, Esch-sur-Alzette (LU); Laura PUCHOT, Esch-sur-Alzette (LU)

(21) Appl. No.: 18/256,804

(22) PCT Filed: Dec. 9, 2021
(86) PCT No.: PCT/EP2021/084927

§ 371 Date: Jun. 9, 2023

(30) Foreign Application Priority Data
Dec. 9, 2020 (LU) LU102316

(51) Int. Cl. C07D 265/16; C08G 73/02

(52) U.S. Cl. CPC C07D 265/16; C08G 73/0233

(57) ABSTRACT

A process for producing a benzoxazine containing free aliphatic hydroxyl groups and monoester comprising the steps of: a) a reaction of a phenolic acid derivative with a monofunctional oligomer or molecule at a temperature of from 80° C. to 200° C., during 12 h-48 h, in a presence of a Bronsted type acid catalyst, resulting in a monophenol terminated oligomer or molecule and b) reaction of the monophenol terminated oligomer or molecule of step a) with a mixture of an amino-alcohol, a primary amine derivative and paraformaldehyde at a temperature range of from 80° C. to 100° C., from 1 h to 48 h, under stirring.

### CROSS-REFERENCE TO RELATED APPLICATIONS

The present invention is the US national stage under 35 U.S.C. § 371 of International Application No. PCT/EP2021/084927 which was filed on Dec. 9, 2021, and which claims the priority of application LU102316 filed on Dec. 9, 2020 the contents of which (text, drawings and claims) are incorporated here by reference in its entirety.

### FIELD

The invention is directed to the field of catalysts for benzoxazine compounds based on transesterification mechanism.

### BACKGROUND

Benzoxazine gives thermosetting properties such as high-temperature and flammability performance, high strength, thermal stability, low water absorption, chemical resistance, low melt viscosities, and near-zero shrinkage. However, they require a lot of time and a high temperature to be polymerized. It impedes their use in many industrial sectors, as for instance for composite elaboration where high production cadence are required. To tackle this drawback, catalysts can be added to decrease the requirements essential for the curing of benzoxazine. Among them lithium, zinc, sodium, or ammonium salts contribute to lower the onset of polymerization (Tₒₙₛₑₜ) to reach for the best cases Tₒₙₛₑₜ=160° C.

However, these catalysts are not covalently linked to the network and can be released with time. In addition, most of them are harmful. Carboxylic acid-containing benzoxazine were also used as catalysts for thermal polymerization of benzoxazines. Other documents mention reaction of benzoxazine-base phenolic resins with strong and weak carboxylic acids and phenols as catalysts.

However, these catalytic systems are not enough stable under heating.

### SUMMARY

The invention has for a technical problem to provide a solution to at least one above mentioned drawback. More specifically, the invention has for generic technical problem to provide a catalytic system for polymerization of benzoxazine monomers.

For this purpose, the invention is directed to a benzoxazine containing free aliphatic hydroxyl groups and a monoester of formula (I)

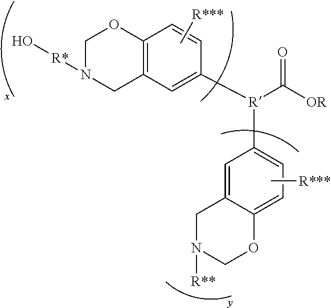

wherein
R is selected from the group consisting of a linear or branched C₁-C₁₂ alkyl or alkoxy group, a linear or branched C₂-C₆ alkenyl or alkylenoxy group, a substituted or unsubstituted linear or branched C₂-C₆ alkynyl group, a cyclo(C₃-C₆ alkyl) group, a heterocyclo(C₃-C₆ alkyl), a linear or branched C₁-C₆ alkyl or C₂-C₆ alkenyl substituted or unsubstituted phenyl group and a (CH₂)ₙ₃-phenyl group, wherein n3 is an integer from 1 to 10;
R′ is selected from the group consisting of at least one of —CH, a C—(CH₂)ₙ₃—CH₃ group, a C—(CH₂)ₙ₃—CH—(CH₃)₂ group, a C—(CH₂)ₙ₃—(CHZ)ₙ₄—(CH₃)₂ group, a C—(CH₂)ₙ₃—(CHZ)ₙ₄—(CH₂)ₙ₃—CH₃ group, a C—(CHZ)ₙ₄—(CH₂)ₙ₃—CH₃ group, a C—(CHZ)ₙ₄—[(CH₂)ₙ₃—CH₃]₂ group, a C-substituted or unsubstituted C₂-C₆ linear or branched alkenyl group, a linear or branched C₁-C₆ alkyl substituted or unsubstituted phenyl or phenyl including at least one hetero atom selected from N, O and S, a C—(CH₂)ₙ₃—C₁-C₆ linear or branched alkyl substituted or unsubstituted phenyl or phenyl including at least one heteroatom selected from N, O and S, a C—(CH₂)ₙ₃—C₁-C₆ linear or branched alkyl substituted or unsubstituted phenyl or phenyl including at least one heteroatom selected from N, O and S—(CH₂)ₙ₃—CH₃, a C—(CH₂)ₙ₃—C₁-C₆ linear or branched alkyl substituted or unsubstituted phenyl or phenyl including at least one heteroatom selected from N, O and S—(CH₂)ₙ₃—CH—(CH₃)₂, a C—(CH₂)ₙ₃—C₁-C₆ linear or branched alkyl substituted or unsubstituted phenyl or phenyl including at least one heteroatom selected from N, O and S—(CH₂)ₙ₃—(CHZ)ₙ₄—(CH₃)₂ group, a C—(CH₂)ₙ₃—C₁-C₆ linear or branched alkyl substituted or unsubstituted phenyl or phenyl including at least one heteroatom selected from N, O and S—(CHZ)ₙ₄—(CH₂)ₙ₃—CH₃ group and a C—(CH₂)ₙ₃—(CHZ)ₙ₄—C₁-C₆ linear or branched alkyl substituted or unsubstituted phenyl or phenyl including at least one heteroatom selected from N, O and S—(CH₂)ₙ₃—CH₃ group, wherein n3 and n4, independently, are an integer from 1 to 10 and Z is selected from the group consisting in a linear or branched C₁-C₆ alkyl or alkoxy group, linear or branched C₂-C₆ alkenyl or alkylenoxy group and a linear or branched C₁-C₆ alkyl or C₂-C₆ alkenyl substituted or unsubstituted phenyl group, and at least one 0 atom is present or not between two adjacent C, or R′ is omitted. “R′ is omitted” means that the ester moiety is directly linked to the aromatic ring, with y=0.
R* is selected from the group consisting of a linear or branched C₁-C₂₀, preferably C₁-C₆, alkyl or alkoxy group, a cyclo(C₃-C₆ alkyl) group, a heterocyclo(C₃-C₆ alkyl) group, wherein the hetero atom is selected from N, S, and O, linear or branched C₂-C₆ alkenyl or alkylenoxy group, substituted or unsubstituted linear or branched C₂-C₆ alkynyl group, a linear or branched C₁-C₆ alkyl or C₂-C₆ alkenyl substituted or unsubstituted phenyl group, a (CH₂)ₙ₃-phenyl group and —(CH₂)ₙ₃—O—(CH₂)ₙ₄, wherein n3 and n4, independently, are an integer from 1 to 10;
R** is the same as R* and further includes a member selected from a O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group and a O-substituted or unsubstituted C₂-C₆ linear or branched alkynyl group, Z being as defined for R′, a —(CH₂)ₙ₃—CEN group, a polycyclic aromatic or heteroaromatic hydrocarbon, such as naphthalene, anthracene, fluorene, phenanthrene, optionally substituted by a linear or branched C₁-C₆ alkyl or alkoxy group, a cyclo(C₃-C₆ alkyl) group, a heterocyclo(C₃-C₆alkyl) group, a linear or branched C₂-C₆ alkenyl or alkylenoxy group, or by a substituted or unsubstituted linear or branched C₂-C₆ alkynyl group, wherein n3 and n4, independently, are an integer from 1 to 10;

R*** is selected from the group consisting in H, OH and a O-linear or branched C₁-C₆ alkyl group, and further includes a linear or branched C₁-C₁₅ alkyl group or a C₂-C₁₅ alkenyl group or

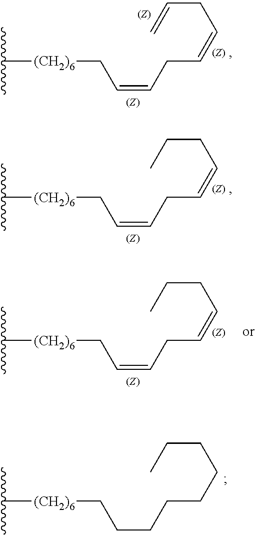

and

x value is of from 0 to 1 and y value is 1−x, preferably of from 0.1 to 1, more preferentially from 0.5 to 1.

In the context of the invention, x and y represent the proportion between benzoxazine groups when prepared from an aminoalcohol and the other amine(s). In other words, x and y can be defined as

$$x = \frac{{n}_{aminoalcohol}}{{n}_{amines}^{total}}$$

$$y = \frac{{n}_{amines}}{{n}_{amines}^{total}}$$

wherein nₐₘᵢₙₑₛ^{total}=nₐₘᵢₙₑₛ+n_{aminoalcohol}, and n_{aminoalcohol} being the number of aminoalcohol per molecules of catalyst, nₐₘᵢₙₑₛ represent the number of amines (excepting the number of aminoalcohol) per molecule of catalyst and nₐₘᵢₙₑₛ^{total} is the total number of amino groups per molecule of catalyst.

The monoester-benzoxazine-of formula (I) are including a hydroxyl group, an ester bond and a benzoxazine ring, combination of which is the essential feature of the invention. The Applicant has shown that the benzoxazine monomers can advantageously lead to the transesterification occurring between the OH and the ester bonds, triggering the polymerization of the benzoxazine. The characteristic tertiary amine of the benzoxazine ring will then catalyze the transesterification reaction, which will catalyze the benzoxazine Ring-Opening Polymerisation (ROP), leading to a polybenzoxazine derivative. It could be considered as a virtuous loop. Consequently, the monoester benzoxazine-containing free aliphatic hydroxyl groups is a catalyst for ROP reaction, as self-polymerisation. In addition, such monoester benzoxazine, once introduced in a traditional and commercial benzoxazine, triggers the polymerization at lower temperature and shorter times. Such monoester benzoxazines involve a green transesterification with high yield, which is solventless and not harmful.

According to the compounds of formula (I), R′ is structure making the bridge between the ester bond and the phenolic ring, and R*** is a substituent of the phenolic ring. It is preferred that R*** is on meta position(s).

R can preferably be selected from the group consisting of a linear or branched C₁-C₄ alkyl or alkoxy group, a linear or branched C₂-C₄ alkenyl or alkylenoxy group, an unsubstituted linear or branched C₂-C₄ alkynyl group, an unsubstituted phenyl group and a (CH₂)ₙ₃-phenyl group, wherein n3 is an integer from 1 to 6.

R′ can preferably be selected from the group consisting of at least one of —CH, a C—(CH₂)ₙ₃—CH₃ group, a C—(CH₂)ₙ₃—CH—(CH₃)₂ group, a C—(CH₂)ₙ₃—(CHZ)ₙ₄—(CH₃)₂ group, C—(CH₂)ₙ₃—(CHZ)ₙ₄—(CH₂)ₙ₃—CH₃ group, C—(CHZ)ₙ₄—(CH₂)ₙ₃—CH₃ group, a C—(CHZ)ₙ₄—[(CH₂)ₙ₃—CH₃]₂ group, a C-substituted or unsubstituted C₂-C₄ linear or branched alkenyl group, an unsubstituted phenyl or phenyl including at least one heteroatom selected from N, O and S, a C—(CH₂)ₙ₃-unsubstituted phenyl or phenyl including at least one heteroatom selected from N, O and S, a C—(CH₂)ₙ₃-unsubstituted phenyl or phenyl including at least one heteroatom selected from N, O and S—(CH₂)ₙ₃—CH₃, a C—(CH₂)ₙ₃-unsubstituted phenyl or phenyl including at least one heteroatom selected from N, O and S—(CH₂)ₙ₃—CH—(CH₃)₂, a C—(CH₂)ₙ₃-unsubstituted phenyl or phenyl including at least one heteroatom selected from N, O and S—(CH₂)ₙ₃—(CHZ)ₙ₄—(CH₃)₂ group, a C—(CH₂)ₙ₃-unsubstituted phenyl or phenyl including at least one heteroatom selected from N, O and S—(CHZ)ₙ₄—(CH₂)ₙ₃—CH₃ group and a C—(CH₂)ₙ₃—(CHZ)ₙ₄— unsubstituted phenyl or phenyl including at least one heteroatom selected from N, O and S—(CH₂)ₙ₃—CH₃ group, wherein n3 and n4, independently, are an integer from 1 to 6 and Z is selected from the group consisting in a linear or branched C₁-C₄ alkyl or alkoxy group, linear or branched C₂-C₄ alkenyl or alkylenoxy group and an unsubstituted phenyl group, and at least one O atom is present or not between two adjacent C.

R* can preferably be selected from the group consisting of a linear or branched C₁-C₆, preferably C₁-C₆, alkyl or alkoxy group, a linear or branched C₂-C₄ alkenyl or alkylenoxy group, an unsubstituted linear or branched C₂-C₄ alkynyl group, an unsubstituted phenyl group, a (CH₂)ₙ₃-phenyl group and —(CH₂)ₙ₃—O—(CH₂)ₙ₄, wherein n3 and n4, independently, are an integer from 1 to 6;

Preferably, R** is the same as R* and can further include a member selected from O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group and a O-substituted or unsubstituted C₂-C₄ linear or branched alkynyl group, Z being as defined above, a —(CH₂)ₙ₃—C≡N group, a cyclo(C₃-C₄ alkyl) group, a heterocyclo(C₃-C₄ alkyl) group, a polycyclic aromatic or heteroaromatic hydrocarbon, wherein the hetero atom is selected from N, S, and O, such as naphthalene, anthracene, fluorene, furane, which can optionally be substituted by a linear or branched C₁-C₄ alkyl or alkoxy group, a linear or branched C₂-C₄ alkenyl or alkylenoxy group, or by a substituted or unsubstituted linear or branched C₂-C₄ alkynyl group, wherein n3 and n4, independently, are an integer from 1 to 6;

R*** can preferably be selected from the group consisting in H, OH and a O-linear or branched C₁-C₄ alkyl group, and can further include a linear or branched C₁-C₁₀ alkyl group or C₂-C₁₀ alkenyl group or

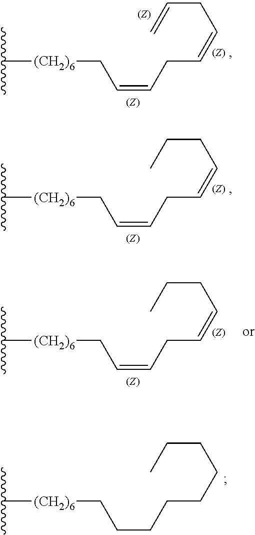

More preferably, R can be selected from the group consisting of groups —CH₃, —(CH₂)ₙ₃—CH₃, —(CH₂)ₙ₃—CH—[(CH₂)ₙ₃—CH₃]₂, —C(CH₃)₃, —(CH₂)ₙ₃—(C₆H₅), —(CH₂)ₙ₃—CH═CH₂ and —(CH₂)ₙ₃—C≡CH, wherein n3 is an integer from 1 to 5.

More preferably, R′ can be selected from the group consisting of groups —CH, C(CH₃), —C—CH(CH₂CH₃), —C(CH₂CH₂CH₃), —C—CH₂(CH₂)₃CH₃, —C—CH₂(CH₂)₄CH₃, —C(C₆H₅), —C(CH₃)CH₂, C(CH₃)CH₂CH₂ and —C(C₆H₅)CH₂—CH₃.

More preferably, R* can be selected from the group consisting of groups —CH₃, —(CH₂)ₙ₃—CH₃, —(CH₂)ₙ₃—CH—[(CH₂)ₙ₄—CH₃]₂, —C(CH₃)₃, (CH₂)ₙ₃—(C₆H₅), —(CH₂)ₙ₃—CH═CH₂, —(CH₂)ₙ₃—C≡CH, —(CH₂)ₙ₃—O—(CH₂)ₙ₄ wherein n3 and n4 independently are integer from 1 to 4, phenyl, and —(CH₂)₃-phenyl.

More preferably, R** can be the group R*, or can be selected from the group consisting of groups CH₃, —(CH₂)ₙ₃—CH₃, —(CH₂)ₙ₃—CH—[(CH₂)ₙ₄—CH₃]₂, —C(CH₃)₃, (CH₂)ₙ₃—(C₆H₅), —(CH₂)ₙ₃—CH═CH₂, —(CH₂)ₙ₃—C≡CH, O—(CH₂)ₙ₃—C≡CH, O—(CH₂)ₙ₃—C≡N, (CH₂)ₙ₃—C≡N, and —(CH₂)ₙ₃-substituted or unsubstituted furan, phenyl, and wherein n3 and n4, independently, are integer from 1 to 4.

R*** can preferably be selected from the group consisting in H, OH and a O-linear or branched C₁-C₃ alkyl group, and can further include linear or branched C₁-C₆ alkyl group or C₂-C₆ alkenyl group or

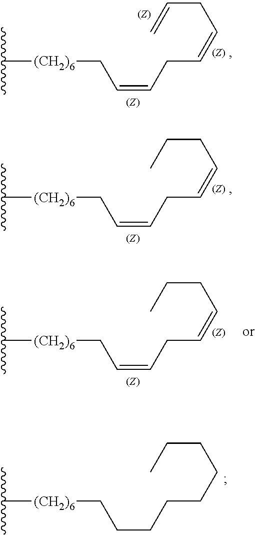

More preferably R*** is H.

The depicted R, R′, R*, R**, R*** and combination thereof can be used independently one from the other.

The expression “substituted” as defined above, relates to the presence of some linear or branched alkyl groups in C₁-C₆.

The invention also relates to a process (1) for producing a benzoxazine-containing free aliphatic hydroxyl groups and monoester of formula (I) comprising the following steps of:
a) reaction of a phenolic acid derivative of formula (II), comprising at least one R*** group,

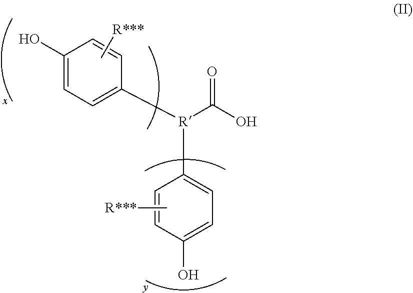

with a monofunctional oligomer or molecule of formula (III)

R—OH (III)

at a temperature of from 80° C. to 200° C., during 12 h-48 h, in a presence of a Bronsted type acid catalyst, resulting in a phenol terminated oligomer or molecule of formula (IV)

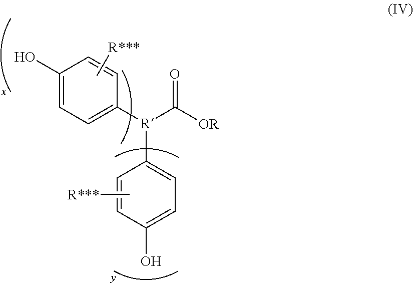

and
b) reaction of the phenol terminated oligomer or molecule of formula (IV) with a mixture of
   an amino-alcohol of formula (V):

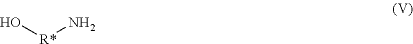

a primary amine derivative of formula (VI),

R**—NH₂ (VI), and
paraformaldehyde of formula (VII)

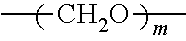

m m=8-100

at a temperature range of from 80° C. to 100° C., from 1 h to 48 h, under stirring, wherein R, R′, R*, R**, R***, x and y are, independently, as defined above, with the proviso that when at least one R*** of the phenolic acid derivative is in ortho position with regard to —OH group, then R*** is H.

The monoester benzoxazine-containing free aliphatic hydroxyl groups of formula (I) is synthesized in two stages. The first step (step a)) corresponds to a Fischer esterification between a monofunctional molecule or oligomer terminated with an aliphatic hydroxyl group and a phenolic acid derivative in presence of Bronsted type acid catalyst introduced in catalytic amount. The reagents are reacted together at 80° to 200° C. and under mechanical stirring for 12-48 hours. The second step (step b)) corresponds to a Mannich condensation of the freshly prepared ester and phenol functionalized molecule with paraformaldehyde, a linear bifunctional molecule amino-alcohol, and a primary amine derivative, wherein x is between 0 and 1, and y=1−x.

The monoester-benzoxazine of the invention is advantageously suited for obtaining polybenzoxazine derivatives by a polymerization involving the benzoxazine ring opening and a self-polymerisation under heat.

Consequently, the Applicant has shown that the monoester-benzoxazines can advantageously lead to the transesterification occurring between the OH and the ester bonds trigger the opening of benzoxazine rings, leading to the formation of a tertiary amine. This tertiary amine will then catalyse the transesterification reaction, which will catalyse the benzoxazine Ring-Opening Polymerisation (ROP), leading to a polybenzoxazine derivative.

The Applicant has shown that the specific starting reactants are providing a benzoxazine monoester containing free aliphatic hydroxyl groups, which in turn, after polymerization, is giving the polybenzoxazine derivatives comprising polymerized benzoxazine.

The benzoxazine ring, obtained from the reaction of the specific derivatives which allows the material to be cross-linked (processed) upon heating, helps the reprocessing thanks to the exchangeable and reversible ester bonds, and free aliphatic hydroxyl groups. Also, the benzoxazine ring moiety gives thermosetting properties such as high-temperature and flammability performance, high strength, thermal stability, low water absorption, chemical resistance, low melt viscosities, and near-zero shrinkage.

The phenolic acid derivative (formula (II)) can include at least one R*** group, more preferably of from 1 to 4, related to the substitution of the phenolic ring, and the R group related to the nature of the bridge between the ester bonds and the phenolic ring.

It is advantageous that the phenolic acid derivative (formula (II)) bears R*** groups that does not interfere with the phenolic ortho-position to avoid steric hindrance that can adversely impact the kinetic of step a) or the oxazine ring closure of step b).

Accordingly, R*** groups can then be advantageously selected to bear short chain groups, as previously defined, with the proviso that R*** in phenolic ortho-position is H.

In some embodiments, there could be two phenolic ortho-positions, each of which is H for the R*** group.

As an example, when the phenolic acid derivative is a monophenol, then x=1 and y=0, whereas when the phenolic acid derivative is a diphenol, then x=0, 5 and y=0.5.

In the context of the invention, “derivative” in “phenolic acid derivative” means a compound bearing phenol and carboxylic acid moieties. Accordingly, “phenolic acid derivative” also means an organic compound bearing phenol and carboxylic acid moieties without being limitative.

The phenolic acid derivative can be more preferably selected from the group consisting of mono-, di-, tri-hydroxybenzoic acid derivatives, anacardic acid derivatives, hydroxycinnamic acid derivatives, aliphatic X-hydroxyphenyl acid derivatives, wherein X is 2-4 and aliphatic diphenolic acid derivatives, or mixtures thereof.

Most preferred aliphatic mono-, di-, tri-hydroxybenzoic acid derivatives can be of formula (VIII)

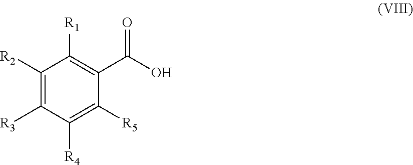

wherein R′ is omitted, and the R₁ to R₅ groups corresponding to R***, and one among R₁-R₅ is a hydroxyl group, then at least one H is in phenolic ortho-position, the rest being defined above.

Especially, in formula (VIII), at least one combination of R₁ to R₅ can be selected from the group consisting of:
R₁=OH, R₂=H, R₃=R₄=R₅=H or CH₃ or CH₂—CH₃ or CH₂—CH₂CH₃ or CH₂—CH(CH₃)₂,
R₂=OH, R₁=R₃=H, R₄=R₅=H or CH₃ or CH₂—CH₃ or CH₂—CH₂CH₃ or CH₂—CH(CH₃)₂,
R₃=OH, R₂=R₄=H, R₁=R₅=H or CH₃ or CH₂—CH₃ or CH₂—CH₂CH₃ or CH₂—CH(CH₃)₂,
R₄=OH, R₃=R₅=H, R₁=R₂=H or CH₃ or CH₂—CH₃ or CH₂—CH₂CH₃ or CH₂—CH(CH₃)₂,
R₅=OH, R₁=H, R₂=R₃=R₄=H or CH₃ or CH₂—CH₃ or CH₂—CH₂CH₃ or CH₂—CH(CH₃)₂.

Most preferred anacardic acid derivatives can be of formula (IX),

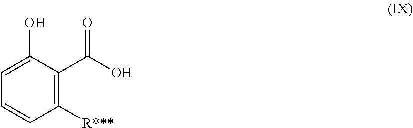

wherein R′ is omitted, and R*** is

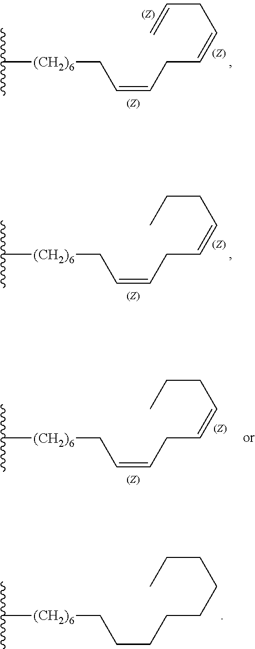

Most preferred hydroxycinnamic acid derivatives can be of formula (X)

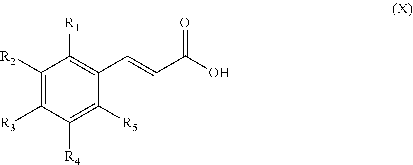

wherein R₁ to R₅ are corresponding to R***, and one among R₁-R₅ is a hydroxyl group and at least one H being in phenolic ortho-position, the rest being H and, optionally an aliphatic alkyl or alkoxy group of C₁-C₆.

Most preferred aliphatic X-hydroxyphenyl acid derivatives can be selected from the group consisting of aliphatic hydroxyphenyl acids (X=1) di-hydroxyphenyl acids (X=2), aliphatic tri-hydroxyphenyl acids (X=3) and aliphatic tetra-hydroxyphenyl acids (X=4), or mixtures thereof, of formula (XI)

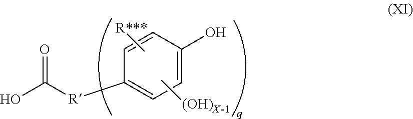

wherein R′ and R*** are as previously defined.

The number of R*** in the ring is depending on the number of hydroxyl groups in the ring, and at least one R***, preferably of from 1 to 3, is H towards the phenolic ortho-position, and the integer q is comprised between 1 and 3.

Most preferred diphenolic acid derivatives are of formula (XII)

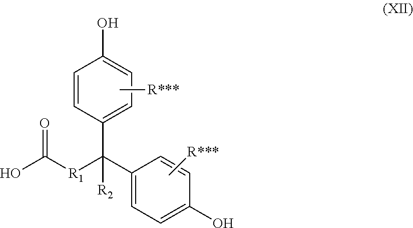

wherein
in the formula, —R₁—C—R₂— moiety is R′;
on each respective phenolic cycle, at least one R***, preferably of from 1 to 3, is H towards the phenolic ortho-position, and otherwise R*** is as defined previously, and R₂ is selected from the group consisting of (CH₂)ₙ₅CH₃, (CH₂)ₙ₄-(aliphatic C₁-C₆ aliphatic alkyl or alkoxy substituted or unsubstituted phenyl group), wherein n5 is an integer from 1 to 12, preferably from 1 to 10, more preferably from 1 to 6, and (CH₂)ₙ₅(CH(CH₃)₂), and
R₁ is selected from the group consisting of (CH₂)ₙ₆, wherein n6 is an integer from 1 to 3, CH(CH₂)ₙ₆(CH₃), CH(CH(CH₃)₂) and C(CH₃)₂, (CH₂)ₙ₆ being the most preferred to lower the steric hindrance.

Most preferred is the 4,4-Bis(4-hydroxyphenyl)valeric acid (VA or DPA).

The monofunctional oligomer or molecule of formula (III) is an alcohol derivative, R—OH. R is as defined above.

The Bronsted acid type catalyst are those commonly used for a Fischer esterification include para-toluene sulfonic acid (p-TSA), anhydrous chlorohydric acid (HCl), phosphoric acid (H₃PO₄), methanoic acid (CH₃—CO₂H), sulfuric acid, tosylic acid, and Lewis acids such as scandium(III) triflate. The content of catalyst can typically be of from 0.5 wt % to 2 wt %.

The step a) can advantageously be carried out at a temperature in the range of 80° C. to 150° C., most preferably of from 100° C. to 140° C. for the best synthesis yields of higher than 95%, the chosen temperature being dependent on the nature of the reactants, i.e. the melting temperature of the reactant medium.

Advantageously, step a) is performed of from 12 h to 24 h for the highest yield of at least 95%, and the duration is based on the kinetic of the reaction.

The respective stoichiometry of starting reactants on step a), phenolic acid derivative: monofunctional molecule or oligomer can preferably be 1.0-3.0 eq.:1.0 eq, resulting in an 1.0 eq. of phenol terminated oligomer or molecule.

The second step of the process, step b), corresponds to a Mannich condensation type reaction of the phenol terminated oligomer or molecule of step a) (formula (IV)) with an amino-alcohol (formula (V)), a primary amine derivative of formula (VI) and the paraformaldehyde, optionally in presence of a catalyst. Thus, since step b) does not require the use of an external catalyst, step b) is implemented in an easier way.

Advantageously, the amino-alcohol of formula (V) includes R* group, a linear amino-alcohol with a primary amine moiety and an aliphatic hydroxyl moiety for obtaining with the highest yield and the best reaction conditions the oxazine ring.

The amino-alcohol of formula (V) can be more preferably selected from the group consisting of 2-aminoethanol, 2-(2-aminoethoxy)ethanol, 2-amino-2-methylpropanol, 5-aminopentan-1-ol, heptaminol and diglycolamine, or mixtures thereof.

The primary amine derivative includes the R** group, as defined above.

In the context of the invention, “derivative” in “primary amine derivative” means a compound bearing a primary amine moiety. Accordingly, “primary amine derivative” also means an organic compound bearing a primary amine group without being limitative.

Primary amine derivatives bear R** having the definition of R* and can be further selected from the group consisting in allylamine, methylamine, ethylamine, propylamine, butylamine, isopropylamine, hexylamine, cyclohexylamine, stearylamine, 2-aminofluorene, aminophenyl acetylene, propargyl ether aniline, 4-aminobenzonitrile, furfurylamine and aniline, or mixtures thereof.

The temperature range of step b) can preferably be of from 80° C. to 95° C., more allowing to obtain the highest conversion yields of at least 75%.

Advantageously, step b) is performed from 1 h to 3 h, for the highest yield of at least 75%.

One advantage of the invention, is that step b) is performed without any catalyst.

The respective stoichiometry of starting reactants on step b), phenol terminated oligomer or molecule:amino-alcohol:primary amine derivative:paraformaldehyde can preferably be 1.0 eq.:x(1.0 eq-18.0 eq): y(1.0 eq-18.0 eq):2.0-36.0 eq, resulting in an 1.0 eq. of the monoester-benzoxazine, wherein x and y are as previously defined. It is also assumed that the higher is x, the more efficient is the ROP.

The specific range stoichiometry is depending on the respective equivalent proportion of the amino-alcohol and of the primary amine derivative. It should be pointed out that there is a minimal quantity required for the reaction to occur. For instance, the relative molar % of amino-alcohol vs the relative molar % of primary amine derivative is 10 molar % vs 90 molar % respectively. It also means that primary amine can be omitted (0 molar %) and amino-alcohol can only be used (100 molar %). Besides, the selected stoichiometry ranges of both amino-alcohol/amine and paraformaldehyde preferably avoids the formation of either reaction linear and/or aliphatic by-products, such as oxazolidine, triaza derivatives, or condensation derivatives.

Preferentially, the whole process is performed with bio-based reactants.

The monoester-benzoxazine synthesis can most preferably be solventless, even though a solvent could be added for the dissolution of starting reactants. The process involves a one-step synthesis, which is one of the advantages of the invention.

Advantageously, the whole synthesis can generally not require any further monomer purification for the invention to be implemented. However, the purification of the monomer, if needed, can be performed by any known technic (vacuum, distillation etc.) The reaction mixtures of both steps a) and b) are stirred using a classical mechanical stirrer, or any non-limitative means.

The process can be implemented by any known means known to the one skilled in the art, using appropriate vessel either at lab scale or at industrial scale.

The invention also relates to a benzoxazine Ring-Opening Polymerisation (ROP) catalyst comprising a benzoxazine containing free aliphatic hydroxyl groups monoester of formula (I).

The Applicant has shown that the monoester-benzoxazines can advantageously lead to the transesterification occurring between the OH and the ester bonds, triggering the polymerization of the benzoxazine, leading to the formation of a tertiary amine. This tertiary amine will then catalyse the transesterification reaction, which will catalyse the benzoxazine Ring-Opening Polymerisation (ROP), leading to a polybenzoxazine derivative. It could be considered as a virtuous loop.

The invention also relates to the use of a benzoxazine containing free aliphatic hydroxyl groups and monoester of formula (I) of the invention or as obtainable by the process (1) or of an ester containing benzoxazine monomer of formula (XX)

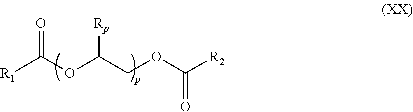

wherein
R₁ is

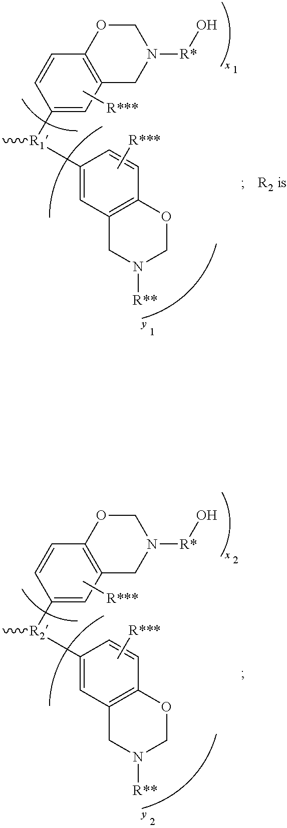

and

Rₚ is selected from the group consisting of H, a linear or branched C₁-C₆, preferably C₁-C₄, alkyl or alkoxy group, a linear or branched C₂-C₆, preferably C₂-C₄, alkenyl or alkylenoxy group, a substituted or unsubstituted linear or branched C₂-C₆, preferably C₂-C₄, alkynyl group, a linear or branched C₁-C₆, preferably C₁-C₄, alkyl or C₂-C₆, preferably C₂-C₄, alkenyl substituted or unsubstituted phenyl group and

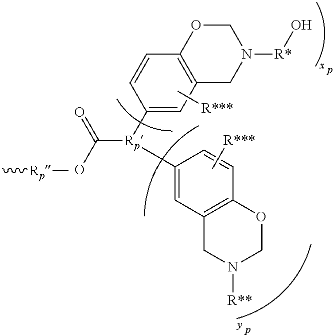

wherein
R₁ and R₂ of formula (XX) are identical or different;
x₁, x₂ and xₚ, independently, are of from 0 to 1 and are not together 0;
y₁=1−x₁; y₂=1−x₂; yₚ=1−xₚ;
p is 1-100;
R₁′, R₂′, and Rₚ′, independently, are selected from the group consisting of a —C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group;
Rₚ″ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group and a linear or branched C₁-C₆ alkyl or C₂-C₆ alkenyl substituted or unsubstituted phenyl group;
R*, R** and R*** are, independently, as defined above,

as a catalyst for benzoxazine polymerization.

Values of x₁, x₂ and xp, independently, are of from 0 to 1, and are not together 0, preferably of from 0.1 to 1, more preferentially of from 0.5 to 1, and y₁, y₂, and yₚ values are, respectively and independently, 1−x₁, 1−x₂ and 1−xₚ. In some embodiments, x₁ and x₂ may not be together 0.

x₁, x₂, xₚ and y₁, y₂, yₚ represent the proportion between benzoxazine groups when prepared from an aminoalcohol and the other amine(s). In other words, x₁, x₂, xₚ and y₁, y₂, yₚ can be defined as

$${x}_{1} = \frac{{n}_{aminoalcohol ( R 1 )}}{{n}_{amines ( R 1 )}^{total}}$$

$${x}_{2} = \frac{{n}_{aminoalcohol ( R 2 )}}{{n}_{amines ( R 2 )}^{total}}$$

$${x}_{p} = \frac{{n}_{aminoalcohol ( Rp )}}{{n}_{amines ( Rp )}^{total}}$$

$${y}_{1} = \frac{{n}_{amines ( R 1 )}}{{n}_{amines ( R 1 )}^{total}}$$

$${y}_{2} = \frac{{n}_{amines ( R 2 )}}{{n}_{amines ( R 1 )}^{total}}$$

$${y}_{p} = \frac{{n}_{amines ( Rp )}}{{n}_{amines ( Rp )}^{total}}$$

wherein n_{amine(R1)}^{total}+n_{aminoalcohol(R1)}, and n_{aminoalcohol(R1)} being the number of aminoalcohol per R₁ group, n_{amines(R1)}, represent the number of amines (excepting the number of aminoalcohol) per group R₁ and n_{amine(R1)}^{total}=n_{amines(R1)}+n_{aminoalcohol(R1)} is the total number of amino groups per group R₁;

wherein n_{amine(R2)}^{total}=n_{amines(R2)}+n_{aminoalcohol(R2)}, and n_{aminoalcohol(R2)} being the number of aminoalcohol per R₂ group, n_{amines(R2)} represents the number of amines (excepting the number of aminoalcohol) per group R₂ and n_{amine(R2)}^{total}=n_{amines(R2)}+n_{aminoalcohol(R2)} is the total number of amino groups per group R₂

wherein n_{amines(Rp)}^{total}=n_{amines(Rp)}+n_{aminoalcohol(Rp)}, and n_{aminoalcohol(Rp)} being the number of aminoalcohol per Rp group, n_{amines(Rp)} represents the number of amines (excepting the number of aminoalcohol) per group Rₚ and n_{amine(Rp)}^{total}=n_{amines(Rp)}+n_{aminoalcohol(Rp)} is the total number of amino groups per group Rₚ.

The Applicant has surprisingly shown that such monoester-benzoxazine of formula (I) and/or such ester containing benzoxazine monomer of formula (XX) are the best suited for ROP of benzoxazine. Advantageously, the onset of the thermal polymerization of benzoxazine ring is lowered with the use of such catalyst(s).

Preferably, R₁′, R₂′, and Rₚ′, independently, can be selected from the group consisting of a —C-a linear or branched C₁-C₄ alkyl or alkoxy group, a —C-linear or branched C₂-C₄ alkenyl or alkylenoxy group, a —C-substituted or unsubstituted linear or branched C₂-C₄ alkynyl group, and a —C-linear or branched C₁-C₄ alkyl or C₂-C₄ alkenyl substituted or unsubstituted phenyl group;

Preferably, Rₚ″ can be selected from the group consisting of a linear or branched C₁-C₄ alkyl or alkoxy group, a linear or branched C₂-C₄ alkenyl or alkylenoxy group, a substituted or unsubstituted linear or branched C₂-C₄ alkynyl group and a linear or branched C₁-C₄ alkyl or C₂-C₄alkenyl substituted or unsubstituted phenyl group.

The invention also relates to a process (2) for synthesizing an ester-containing benzoxazine monomer of formula (XX) comprising the following steps consisting of:
a) reacting a phenolic acid derivative of formula (XXI), comprising at least one R*** group on the phenolic ring,

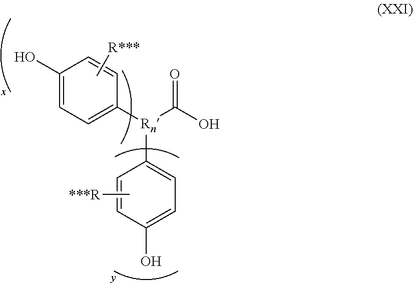

wherein x is of from 0 to 1, and y=1-x,
with a polyfunctional molecule or oligomer of formula (XXII)

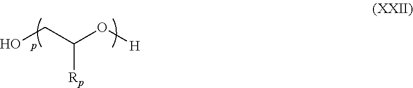

at a temperature of from 25° C. to 200° C., during 1 h-72 h, in the presence of a catalyst of Bronsted acid type, resulting in a phenol terminated oligomer or molecule (compound ((XXIII)),
b) reacting the compound (XXIII) with a mixture of:
   an amino-alcohol of formula (V):

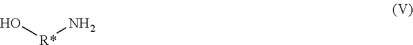

a primary amine derivative of formula (VI), and

R**—NH₂ (VI)
paraformaldehyde of formula (VII)

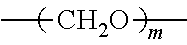

m=8-100

at a temperature range of from 80° C. to 100° C., from 1 h to 10 h, under stirring, for obtaining the compound of formula (XX),

wherein Rₚ, R*, R**, R***, Rₙ′ is R₁′ and R₂′, and p are, independently, as defined above, with the proviso that when at least one R*** of the phenolic acid derivative is in ortho-position with regard to —OH group, then R*** is H.

The process (2) for synthesizing an ester-containing benzoxazine monomer of formula (XX) uses polyfunctional oligomer or molecule of formula (XXII) while the process (1) to synthesize a monoester benzoxazine-containing free aliphatic hydroxyl groups of formula (I) uses monofunctionnal oligomer or molecule of formula (III).

The polyfunctional molecule or oligomer compound of formula (XXII) is of importance for selecting the processing temperature of the benzoxazine polymer.

The compound of formula (XXII) can advantageously have 1-30, better 1-20, especially 1-10, p values, and can represent more preferably, when Rₚ=H, a polyethylene glycol (PEG) with a molecular weight (MW) in the range of from 4 MW of the C₂H₄O unit to 50 MW of the C₂H₄O unit, the MW of the C₂H₄O unit being classically of about 44.05 g/Mol.

It is preferable to use commercially available PEG, for example PEG 200 to PEG 2200, as being easily available.

In the compound of formula (XXII), when Rₚ=H, p values can be of from 1 (ethylene glycol) to 3 (triethylene glycol—TEG).

In some other embodiments, the compound of formula (XXII) can be glycerol (Rₚ=CH₂OH).

The preferred parameters, groups, stoichiometry and implementation conditions for step a) and step b) of the process (2) are the same as those described for step a) and step b) of the process (1), respectively.

The invention also relates to a process (3) for preparing a polybenzoxazine derivative comprising the step of polymerizing a composition comprising a benzoxazine containing free aliphatic hydroxyl groups monoester of formula (I) or as obtainable by the process (1) or an ester-containing benzoxazine monomer of formula (XX) or as obtainable by the process (2), or a mixture thereof, as a benzoxazine catalyst, and comprising of from 0 weight % to 99 weight % of a benzoxazine derivative, different from the benzoxazine catalyst, at temperatures within the range of from 100° C. to 250° C. for 1 h to 24 h, for obtaining polybenzoxazine derivatives.

In the context of the invention, “derivative” in “benzoxazine derivative” or “polybenzoxazine derivatives” means a compound bearing a benzoxazine moiety or issued from a compound bearing a benzoxazine moiety.

The monoester benzoxazine containing free aliphatic hydroxyl groups of formula (I) or the ester-containing benzoxazine monomer of formula (XX) as the benzoxazine catalyst can each react on itself, like the mixture thereof too, to produce the polybenzoxazine derivative, or react with a second benzoxazine derivative different of the benzoxazine catalyst.

The proportion of each of the benzoxazine containing free aliphatic hydroxyl groups and monoester of formula (I) or the ester-containing benzoxazine monomer of formula (XX) in the composition thereof is not limited. But it can advantageous that the proportion is within the range of 0.5 weight % to 95 weight %, better of from 1 to 50 wt %, most preferably of from 5 to 10 wt %.

According to the process for preparing the polybenzoxazine derivatives of the invention, using the compound (1) or the compound (XX), or mixture thereof, and optionally the benzoxazine derivative different from the benzoxazine catalyst, the polymerization step, which is a curing step, allows the benzoxazine ring to open and to react on itself or with another benzoxazine derivative to form a 3D network. Here, compounds of formula (I) or of formula (XX) as such can act on their self to produce the polybenzoxazine derivatives.

The polymerization duration is depending on the curing temperature and/or on the nature of the ester-containing benzoxazine monomer. The polymerization temperature is selected for a given monomer to be higher than the temperature needed to synthesize the monomer. Generally, the higher the polymerization temperature, the shorter the curing duration. For example, when the temperature of the polymerization is 250° C., the curing duration can be of at least 1 h, and for a polymerization temperature of 100° C., the curing duration can be of no more than 24 h. Preferably, the curing temperature can be of from 140° C. to 200° C., more preferably of from 140° C. to 180° C., the latter range providing curing duration of from 1.5 h to 3 h, preferably of from 1.5 h to 2.5 h. The polymerization can be performed by any known heating means, such as laser beam and infrared beam.

The process can also include a post-polymerization step consisting of a heating step which can preferably be carried out at higher temperature than that the polymerization heating step.

The benzoxazine derivative different from the catalyst can be the class of compounds selected from the group consisting of:
a 3,4-dihydro-2H-1,3-benzoxazine monomer having the formula

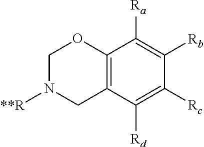

either wherein
Rₐ=CH₃
R_{b} and R_{c} together represent

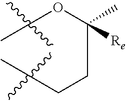

with Rₑ=

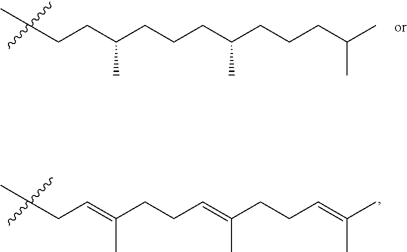 [SMILES: *CC[C@@H](C)CCC[C@@H](C)CCCC(C)C ; *C/C=C(\C)CC/C=C(\C)CCC=C(C)C]

R_{d}=H, or CH₃,
or wherein
R_{d} and R_{c} together represent

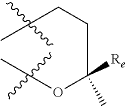

with Rₑ

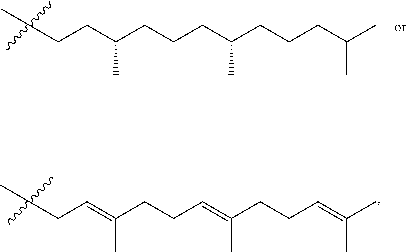 [SMILES: *CC[C@@H](C)CCC[C@@H](C)CCCC(C)C ; *C/C=C(\C)CC/C=C(\C)CCC=C(C)C]

and
   R_{b}=Rₐ=CH₃,
or wherein
R_{c}=R_{d}=H,
R_{b}=

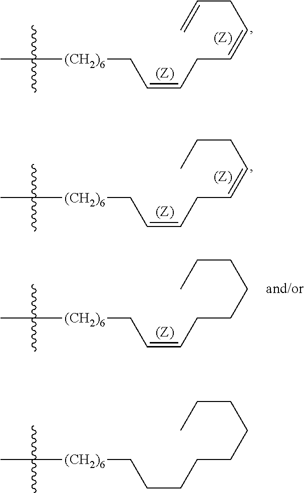

and Rₐ=COOH,
or wherein
Rₐ=H, R_{b}=OH, R_{c}=H
and R_{d}=

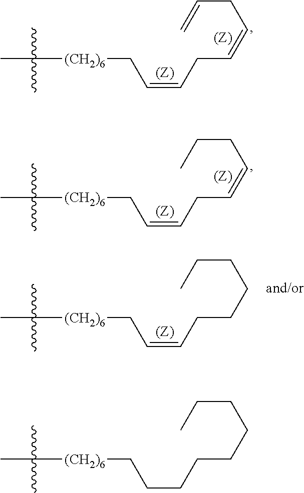

or wherein
Rₐ=H,
and R_{b}=

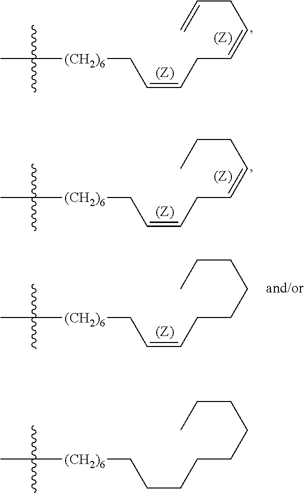

and R_{c}=H, R_{d}=OH,
or wherein
Rₐ=CH₃, R_{b}=OH, R_{c}=H
R_{d}=

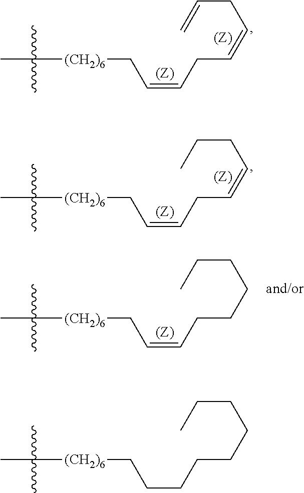

or wherein
Rₐ=R_{c}=R_{d}=H, and
R_{b}=

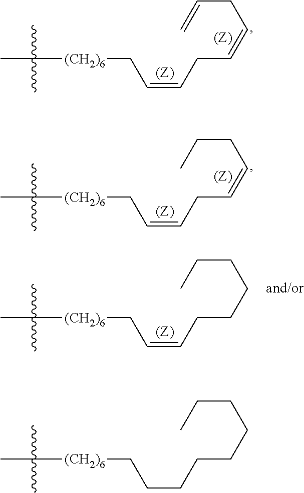

or wherein
R_{b}=R_{d}=H
R_{c}=—CH₂—HC═CH₂ and
Rₐ=OCH₃;
or
R_{b}=R_{d}=H
R_{C}=—CH═HC—CH₃ and
Rₐ=OCH₃,
or a mixture thereof; R** being as defined above;
a compound of formula A, B or C, or mixture thereof, as follows Compound of formula A:
wherein

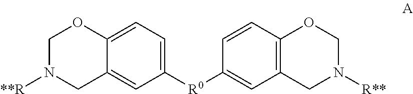

R⁰ is a —CH₂—, —C(CH₃)₂—, SO₂, —C(CF₃)₂—, —C(CH₃)(C₆H₅)—, —C(CH₃)(C₂H₅)—, —C(C₆H₅)₂—, —CHCH₃—, —C₆H₁₀— or —C(CH₃)CH₂CH₂COOH— group;
R** is a —CH₂CH₂OH, vinyl, methyl, ethyl, propyl, isopropyl, butyl, hexyl, cyclohexyl, fluorene, phenylacetylene, phenyl propargyl ether, benzonitrile, furfuryl, phenylene or —(CH₂)₁₇CH₃ group;
compound of formula B

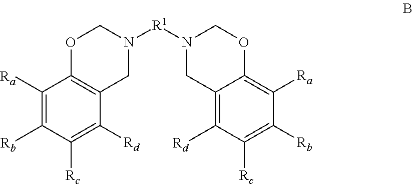

either wherein
R₁ is a —(CH₂)ₙ— group, with n=1-10 or

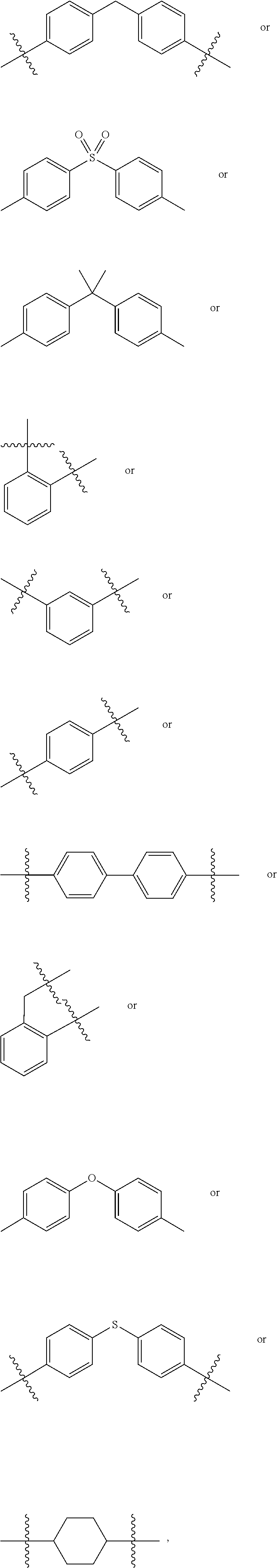

and wherein Rₐ, R_{b},
R_{c}, R_{d} are selected from the group consisting of
Rₐ=R_{b}=R_{c}=R_{d}=H,
Rₐ=OCH₃, R_{b}=R_{d}=H, R_{c}=CH₂CH═CH₂,
Rₐ=OCH₃, R_{b}=R_{d}=H, R_{c}=CH═CHCH₃,
Rₐ=OCH₃, R_{b}=R_{d}=H, R_{c}=CHO,
Rₐ=OCH₃, R_{b}=R_{c}=R_{d}=H,
Rₐ=OCH₃, R_{b}=R_{d}=H, R_{c}=CHO,
Rₐ=OCH₃, R_{b}=R_{d}=H, R_{c}=CH₂CH₂COOH,
Rₐ=R_{b}=R_{d}=H, R_{c}=CH₂CH₂COOH,
Rₐ=R_{b}=R_{d}=H, R_{c}=CH═CHCOOH, and
Rₐ=OCH₃, R_{b}=R_{d}=H, R_{c}=CH═CHCOOH,
or wherein
R_{c}=R_{d}=H,
R_{b}=

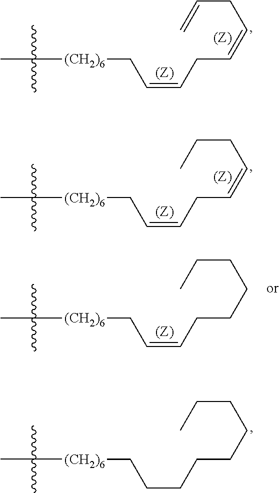

and Rₐ=COOH,
or wherein
Rₐ=H, R_{b}=OH, R_{c}=H
and R_{d}=

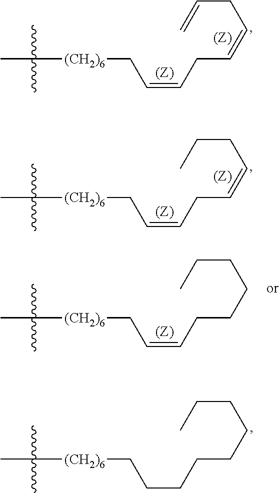

or wherein
Rₐ=H,
and R_{b}=

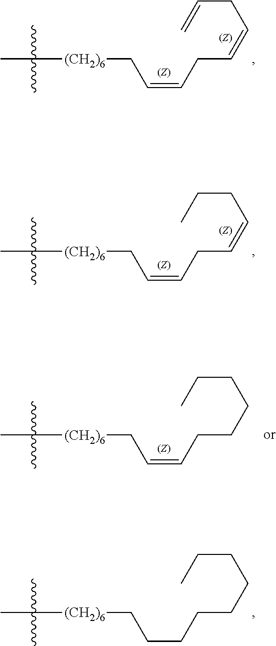

and R_{c}=H, R_{d}=OH,
or wherein
Rₐ=CH₃, R_{b}=OH, R_{c}=H
R_{d}=

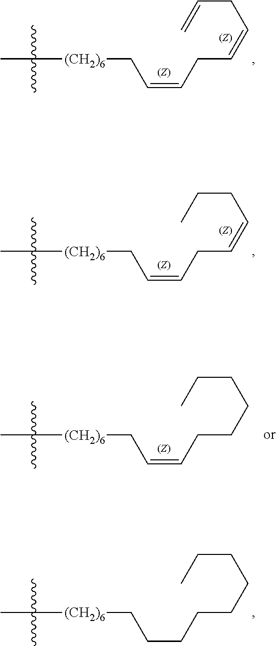

or wherein
Rₐ=R_{c}=R_{d}=H, and
R_{b}=

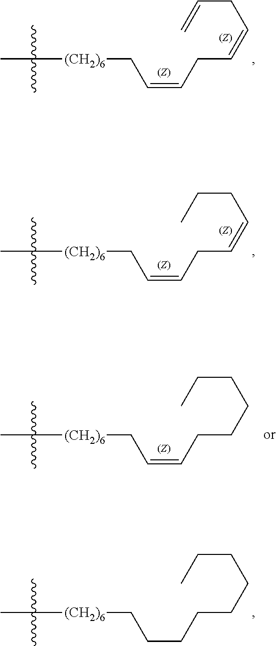

or wherein
Rₐ=R_{c}=H
R_{b}=—CH₂HC═CH₂, and R_{d}=OCH₃;
or
Rₐ=R_{c}=H
R_{b}=—CH═HCCH₃ and
R_{d}=OCH₃;
or Rₐ=R_{b}=R_{d}=H and R_{c}=

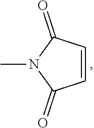

or Rₐ=R_{b}=R_{c}=H and R_{d}=CH₂CH═CH₂
or Rₐ=R_{b}=R_{d}=H and R_{c}=CH₂CH═CH₂
or Rₐ=R_{c}=R_{d}=H and R_{b}=CH₂CH═CH₂;
a compound of formula C

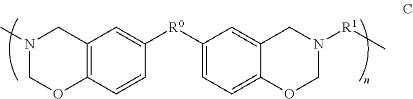

wherein R⁰ is a —CH₂—, —C(CH₃)₂—, SO₂, —C(CF₃)₂—, —C(CH₃)(C₆H₅)—, —C(CH₃)(C₂H₅)—, —C(C₆H₅)₂—, —CHCH₃—, —C₆H₁₀— or —C(CH₃)CH₂CH₂COOH— group;
and either within
n is an integer from 1 to 10;
R¹ is a —(CH₂)ₙ— group, with n=1-10
or

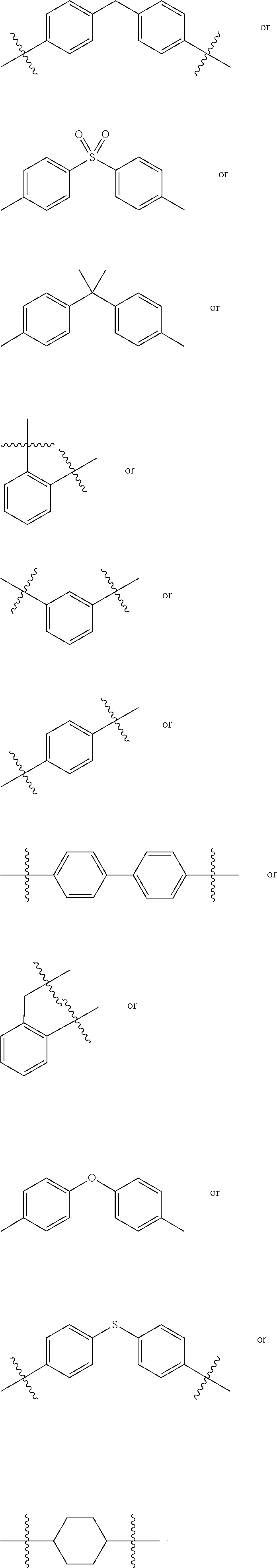

The benzoxazine derivatives: 3,4-dihydro-2H-1,3-benzoxazine monomer, compounds A-C, as described above, are known and synthesis thereof is detailed in WO2020/193293A1, as well as chemical and physical properties of polybenzoxazine derivatives thereof.

The invention also relates to a composition comprising:
a benzoxazine containing free aliphatic hydroxyl groups and monoester of formula (I) or as obtainable by the process (1) or an ester-containing benzoxazine monomer of formula (XX) or as obtainable by the process (2), or a mixture thereof, as a benzoxazine catalyst, and
of from 0 weight % to 99 weight % of a benzoxazine derivative, different of the benzoxazine catalyst and optionally at least one or more additional compounds of organic molecules types not containing benzoxazine moieties.

Preferably, the organic molecules types can be polymers not containing benzoxazine moieties, selected from the group consisting in epoxy resins, bismaleimide resins, phenolic resins or benzoxazine resins, polyurethanes, polyamides, polyolefins, polyesters and rubbers.

The composition can further comprise a material selected from the group consisting of fillers, fibers, pigments, dyes, and plasticizers, or mixture thereof.

Examples of such a material include at least one of carbon fibers, glass fibers, clays, carbon black, silica, carbon nanotubes, graphene, any known means for the thermal or the mechanical reinforcement of composites, or mixtures thereof.

The invention also concerns a use of the polybenzoxazine according to the invention as a reversible adhesive, sealant, coating or encapsulating systems for substrates selected from the group consisting of a metal, polymer, glass and ceramic material. Preferably, the metal and the polymer are as above defined.

### DRAWINGS

Other features and advantages of the present invention will be readily understood from the following detailed description and drawings among them.

FIG. 1 exemplarily shows a synthesis reaction of a monoester with a monofunctionnal phenolic acid for producing pentyl 3-(4-hydroxyphenyl)propanoate (Pent-PA-mea).

FIG. 2 exemplarily shows a synthesis reaction of a monoester with a difunctional phenolic acid for producing pentyl 3-(4-hydroxyphenyl)propanoate (Cyclo-DPA-mea).

FIG. 3a) is an exemplary NMR spectrum of the valeric acid derivative benzoxazine monomer (PEG-DPA-mea) and FIG. 3b) is a NMR spectrum of Pent-PA-mea.

FIG. 4a) exemplarily displays the DSC curves of the PEG-PA-mea, PEG-DPA-mea, PEG-PA-fu, PEG-DPA-fu, FIG. 4b) of the Pent-PA-mea and Pent-PA-fu.

FIG. 5(a) is an exemplary DSC of different mixtures of commercial benzoxazine (ARALDITE® MT 35710): a) monofunctional ester benzoxazine (Pent-PA-mea); FIG. 5b) a cardanol-based benzoxazine (Card-fu, without ester or aliphatic hydroxyl functions) (10° C.min⁻¹, N2).

FIG. 6 is an example of a polybenzoxazine obtained through the use of Pent-PA-mea and Araldite® MT 35710.

FIG. 7 exemplarily shows a synthesis reaction of a monoester with a monofunctional phenolic acid for producing methyl 3-(3-(2-hydroxyethyl)-3,4-dihydro-2Hbenzo[e][1,3]oxazin-6-yl) propanoate (Me-PA-mea).

FIG. 8 exemplarily shows the NMR spectrum of Me-PA-mea ester-containing benzoxazine monomers.

FIG. 9 shows an exemplary synthesis reaction of a monoester with a difunctional phenolic acid for producing methyl 4,4-bis(3-(2-hydroxyethyl)-3,4-dihydro-2H-benzo[e][1,3]oxazin-6-yl)pentanoate (Me-DPA-mea).

FIG. 10 exemplarily shows the NMR spectrum of Me-DPA-mea ester-containing benzoxazine monomers.

FIG. 11(a) exemplarily shows a) the DSC and FIG. 11b) the isothermal rheology monitoring curves of Me-PA/DPA-mea/fu ester-containing benzoxazine monomers.

### DETAILED DESCRIPTION

All chemicals are commercially available and starting compounds, when applies, used as purchased.

### Example 1: Synthesis of Benzoxazine Containing Free Aliphatic Hydroxyl Groups and Monoester (Pent-PA-Mea) with Pentanol, Phloretic Acid, Mono-Ethanol Amine and Paraformaldehyde

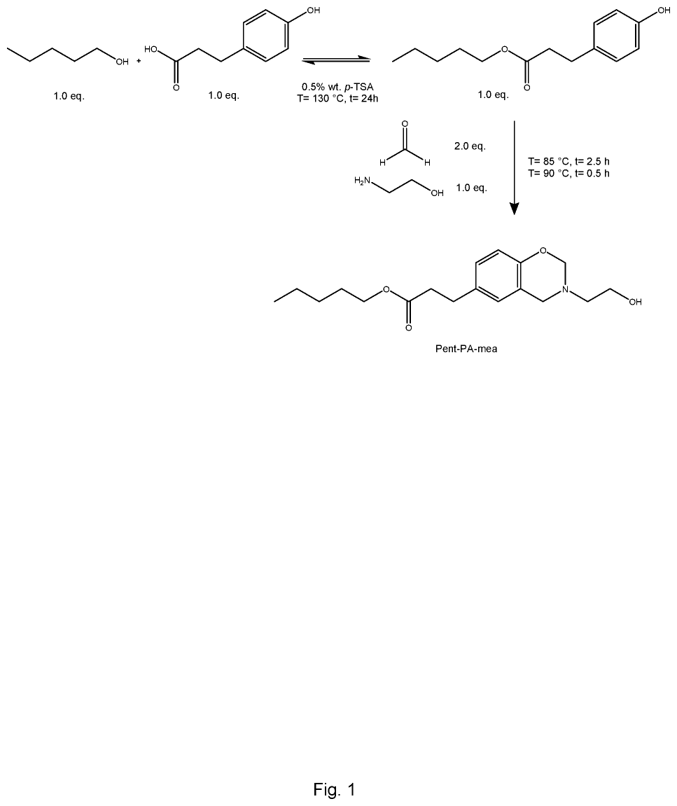

The Pent-PA-mea monoester benzoxazine containing free aliphatic hydroxyl groups was synthesized in two stages (FIG. 1).

The first step, step a), corresponds to a Fischer esterification between pentanol (Pent) (1 eq.) and 3-(4-Hydroxyphenyl)propionic acid (phloretic acid, PA) (1 eq.) in presence of p-toluene sulfonic acid (p-TSA) introduced in catalytic amount (0.5 wt %). The reactants were put together in melt at 130° C. and agitated by mechanical stirring for 24 hours, to provide pentyl 3-(4-hydroxyphenyl)propanoate (Pent-PA) (1 eq.).

The second step, step b), corresponds to a Mannich condensation of Pent-PA (1 eq.) with mono-ethanolamine (mea) (1 eq.) and paraformaldehyde (PFA) (2 eq,). All these reactants were agitated together by mechanical stirring and reacted in melt at 85° C. for 2.5 hours to provide the Pent-PA-mea monoester benzoxazine containing free aliphatic hydroxyl groups, pentyl 3-(3-(2-hydroxyethyl)-3,4-dihydro-2H-benzo[e][1,3]oxazin-6-yl)propanoate.

### Example 2: Synthesis of Benzoxazine Containing Free Aliphatic Hydroxyl Groups Monoester (Cyclo-DPA-Mea) from Cyclohexanol, 4,4-Bis(4-Hydroxyphenyl)Valeric Acid (DPA), Mono-Ethanol Amine and Paraformaldehyde

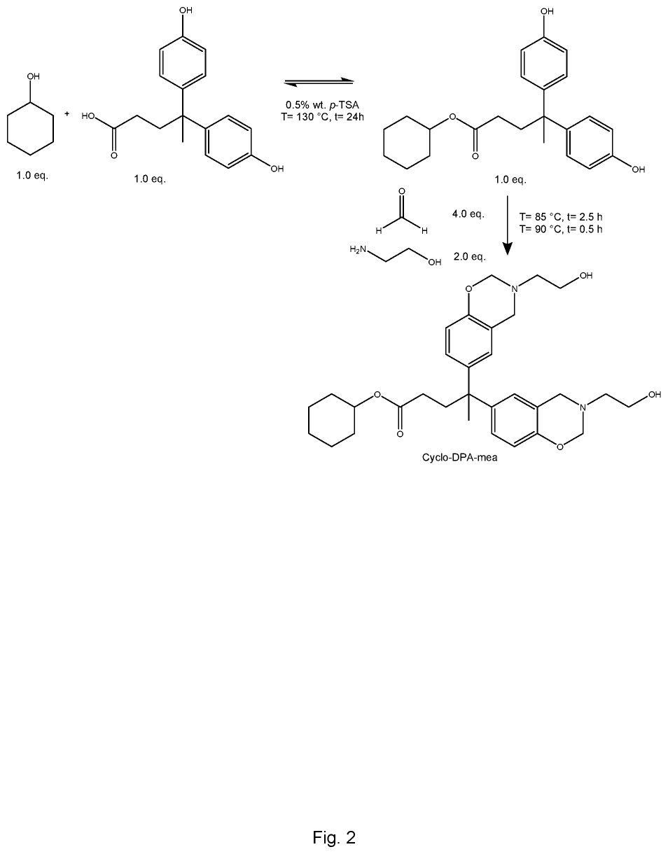

The Cyclo-DPA-mea benzoxazine monoester containing free aliphatic hydroxyl groups was synthesized in two stages (FIG. 2).

The first step, step a), corresponds to a Fischer esterification between cyclohexanol (1 eq.) and 4,4-Bis(4-hydroxyphenyl)valeric acid (DPA) (1 eq.) in presence of p-toluene sulfonic acid (p-TSA) introduced in catalytic amount (0.5 wt %). The reactants were put together in melt at 130° C. and agitated by mechanical stirring for 24 hours, to provide cyclohexyl 4,4-bis(4-hydroxyphenyl)pentanoate (Cyclo-DPA) (1 eq.).

The second step, step b), corresponds to a Mannich condensation Cyclo-DPA (1 eq.), mono-ethanolamine (2 eq.) and paraformaldehyde (4 eq.). All these reactants were agitated together by mechanical stirring and reacted in melt at 85° C. for 2.5 hours followed by 0.5 hours at 90° C. to provide the Cyclo-DPA-mea monoester benzoxazine containing free aliphatic hydroxyl groups, cyclohexyl 4,4-bis(3-(2-hydroxyethyl)-3,4-dihydro-2H-benzo[e][1,3]oxazin-6-yl)pentanoate.

### Example 3; Synthesis of an Ester-Containing Benzoxazine Monomer from 4,4-Bis(4-Hydroxyphenyl)Valeric Acid (DPA) as a Phenolic Acid Derivative

Ester-containing benzoxazine monomer was synthesized in two stages.

The first step, step a), corresponds to a Fischer esterification between polyethylene glycol (PEG) (Mₙ=400 g·mol⁻¹, p=8-9, 1 eq., 10 g) and 4,4-Bis(4-hydroxyphenyl)valeric acid (DPA) (2 eq., 14.32 g) in presence of p-toluene sulfonic acid (p-TSA) introduced in catalytic amount (1 wt %). PEG, DPA and p-TSA were reacted together in melt at 130° C. and agitated by mechanical stirring for 24 hours, to provide 4,4-Bis(4-hydroxyphenyl)valeric ester terminated polyethylene glycol (PEG-DPA).

The second step, step b), corresponds to a Mannich condensation between 4,4-Bis(4-hydroxyphenyl)valeric ester terminated polyethylene glycol (PEG-DPA) (1 eq, 22.8 g), mono-ethanol amine (mea) (4 eq., 5.95 g) and paraformaldehyde (PFA) (8 eq., 5.84 g). All these reactants were agitated together by mechanical stirring and reacted in melt at 85° C. for 2.5 hours followed by 0.5 hours at 90° C. to provide the ester-containing benzoxazine monomer named PEG-DPA-mea, polyethylene glycol terminated 4,4-bis(3-(2-hydroxyethyl)-3,4-dihydro-2H-benzo[e][1,3]oxazin-6-yl)pentanoate.

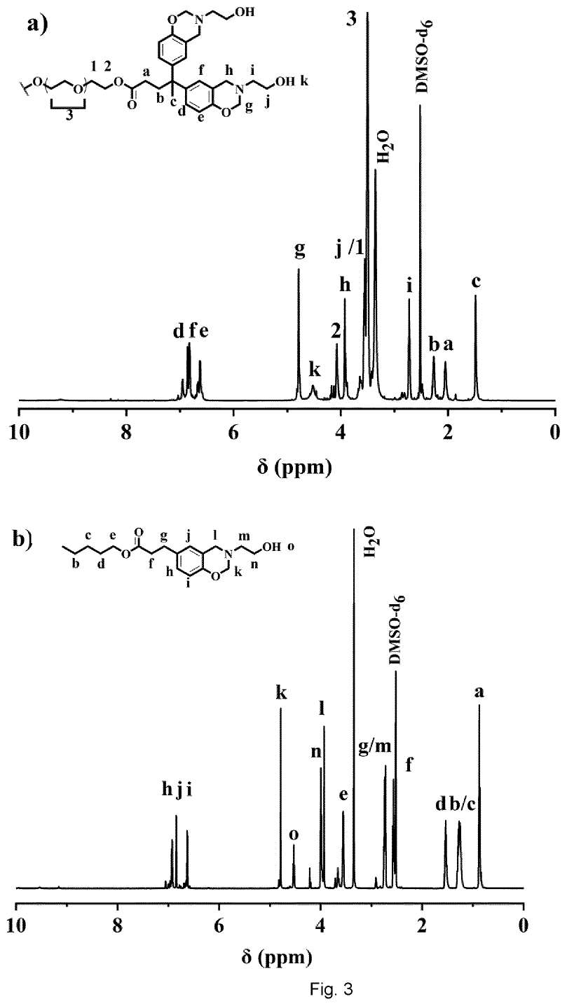

The FIG. 3a) displays the NMR spectrum (AVANCE III HD Bruker spectrometer) of PEG-DPA-mea ester-containing benzoxazine monomer, and FIG. 3b) displays the NMR spectrum of Pent-PA-mea.

### Example 4

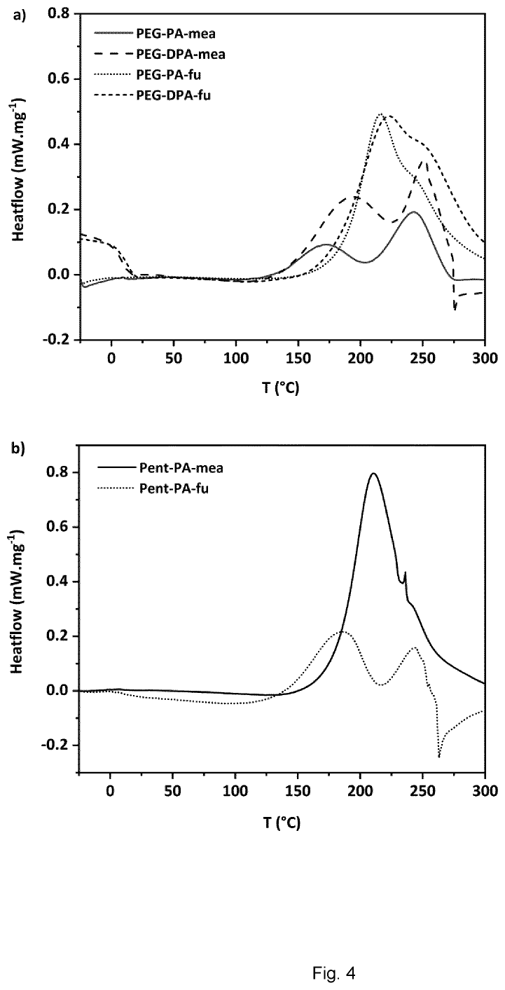

FIG. 4a) displays the DSC curves of the PEG-PA-mea, PEG-DPA-mea, PEG-PA-fu, PEG-DPA-fu, “fu” designating furfurylamine, FIG. 4b) of the Pent-PA-mea and Pent-PA-fu. Conditions: 10° C.min⁻¹, N2 atmosphere.

PEG-PA-mea: polyethylene glycol terminated 3-(3-(2-hydroxyethyl)-3,4-dihydro-2H-benzo[e][1,3]oxazin-6-yl)propanoate.

PEG-PA-fu: polyethylene glycol terminated 3-(3-(furan-2-ylmethyl)-3,4-dihydro-2H-benzo[e][1,3]oxazin-6-yl)propanoate.

PED-DPA-fu: polyethylene glycol terminated 4,4-bis(3-(furan-2-ylmethyl)-3,4-dihydro-2H-benzo[e][1,3]oxazin-6-yl)pentanoate.

PEG-PA-mea, PEG-PA-fu and PEG-DPA-fu ester-containing benzoxazine monomer are obtained as in Example 3, where furfurylamine is used instead of mono-ethanolamine and phloretic acid is used instead of diphenolic acid.

The Pent-PA-fu monomer is obtained as in Example 1, where furfurylamine is used instead of mono-ethanolamine.

Accordingly, FIGS. 4a and 4b are respectively displaying the DSC thermogram of polyfunctional and monofunctional ester containing benzoxazine. The abbreviations “mea” and “fu” correspond respectively to mono-ethanolamine (terminated by free hydroxyl groups) and furfurylamine (terminated by furan groups). For the polyfunctional benzoxazine containing ester and furan groups (FIG. 2a), PEG-PA-fu and PEG-DPA-fu, the DSC thermogram shows a first exothermic peak starting at a temperature of 145° C. with a maximum located around 220° C. This peak is associated to the ring opening of the benzoxazine rings upon heating. The ring opening of the benzoxazine rings occurred at much lower temperature in the case of polyfunctional benzoxazine containing ester and free alcohol groups (FIG. 2a)—PEG-PA-mea and PEG-DPA-mea). The first exothermic peak starts at 105° C. for a maximum located at 175° C.

Transesterification reactions between ester bonds and aliphatic hydroxyl groups promote the thermal ring opening polymerization of benzoxazine monomer. The second exothermic peak corresponds to the degradation the aliphatic ester, observed in both case (mea and fu). In the case of monofunctional benzoxazine (FIG. 2b)), a similar trend is observed. The onset of thermal polymerization appeared at 110 and 140° C. respectively for benzoxazine containing free hydroxyl and furan groups (Pent-PA-mea and Pent-PA-fu).

### Example 5

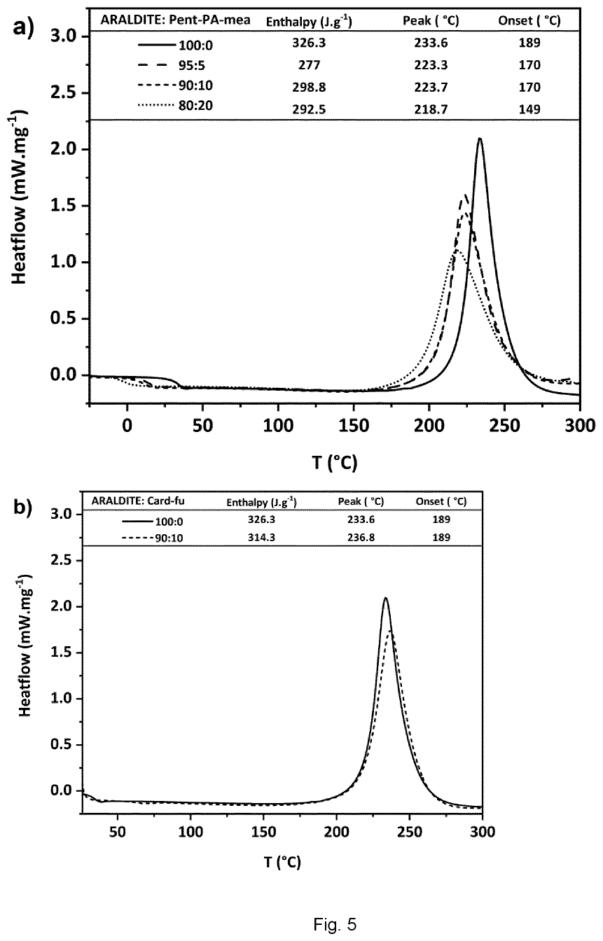

A commercially available benzoxazine monomer (ARALDITE© MT 35710—Huntsmann) was mixed with different ratios of Pent-PA-mea from at 5, 10, 15 and 20% wt. All the mixtures were subjected to DSC studies to evaluate the catalytic activity of this monofunctional benzoxazine (FIG. 5.a). A significant decrease of the onset of thermal polymerization from 189 to 149° C. is observed when the amount of catalyst in the mixtures increases from 0 to 20%. The maximum of the exothermic peak also decreases from 234 to 219° C. in the same condition. The enthalpy of polymerization is not strongly affected by the incorporation of the monofunctional benzoxazine monomer as catalyst (Pent-PA-mea). Therefore, this commercially available benzoxazine monomer (ARALDITE® MT 35710) was mixed with a cardanol-based benzoxazine which contains furfurylamine (terminated by furan groups) but no ester or aliphatic hydroxyl functions (Card-fu) (FIG. 5.b). Card-fu is obtained by reacting cardanol (1 eq), furfurylamine (1 eq) and paraformaldéhyde (2 eq) together during 24 h at 80° C. without solvent. No catalytic activity is observed under these conditions, since the onset values are identical (ratio of Card-fu vs (ARALDITE® MT 35710).

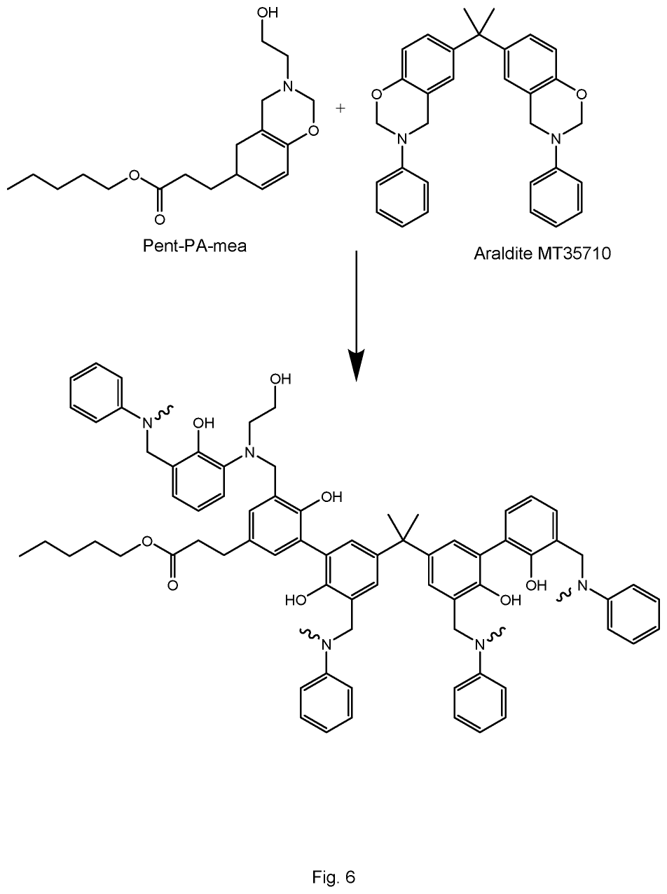

FIG. 6 is an obtained polybenzoxazine through the use of Pent-PA-mea and ARALDITE® MT 35710. The curing of the mixture of Pent-PA-mea and ARALDITE® MT 35710 was 160° C. for 2 hours followed by 1 h at 180° C.

### Example 6: Synthesis of Benzoxazine Containing Free Aliphatic Hydroxyl Groups and Monoester (Me-PA-Mea) with Methanol, Phloretic Acid, Mono-Ethanolamine and Paraformaldehyde

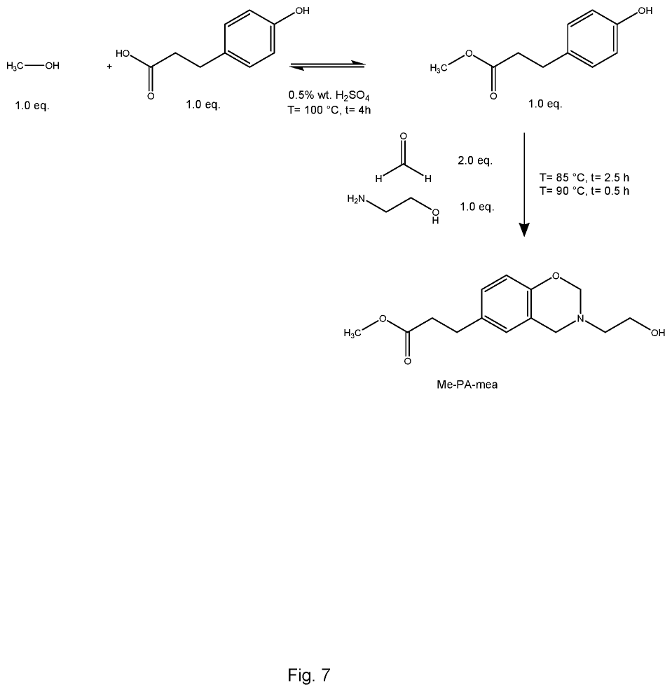

The Me-PA-mea monoester benzoxazine containing free aliphatic hydroxyl groups was synthesized in two stages (FIG. 7). The first step, step a), corresponds to a Fischer esterification between methanol (Me) (1 eq.) and 3-(4-hydroxyphenyl) propionic acid (phloretic acid, PA) (1 eq.) in presence of concentrated sulfuric acid (H₂SO₄) introduced in catalytic amount (0.5 wt %). The reactants were put together in melt at 100° C. under reflux and agitated by magnetic stirring for 4 hours, to provide methyl 3-(4-hydroxyphenyl)propanoate (Me-PA) (1 eq.).

The second step, step b), corresponds to a Mannich condensation of Me-PA (1 eq.) with mono-ethanolamine (mea) (1 eq.) and paraformaldehyde (PFA) (2 eq,). All these reactants were agitated together by mechanical stirring and reacted in melt at 85° C. for 2.5 hours followed by 0.5 hours at 90° C. to provide the Me-PA-mea monoester benzoxazine containing free aliphatic hydroxyl groups, methyl 3-(3-(2-hydroxyethyl)-3,4-dihydro-2Hbenzo[e][1,3]oxazin-6-yl)propanoate.

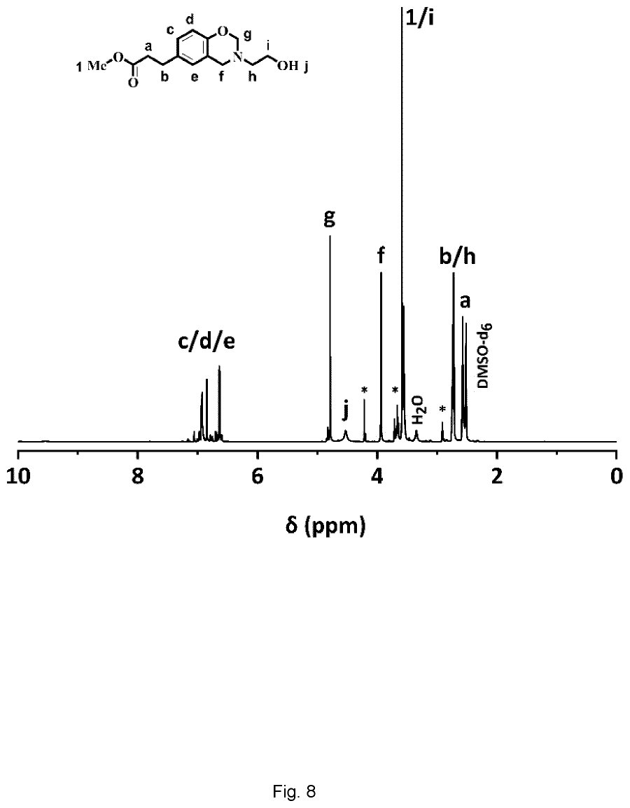

The FIG. 8 is displaying the ¹H NMR spectrum (AVANCE III HD Bruker spectrometer) of Me-PA-mea ester-containing benzoxazine monomers.

### Example 7: Synthesis of Benzoxazine Containing Free Aliphatic Hydroxyl Groups Monoester (Me-DPA-Mea) from Methanol, 4,4-Bis(4-Hydroxyphenyl)Valeric Acid (DPA), Mono-Ethanolamine and Paraformaldehyde

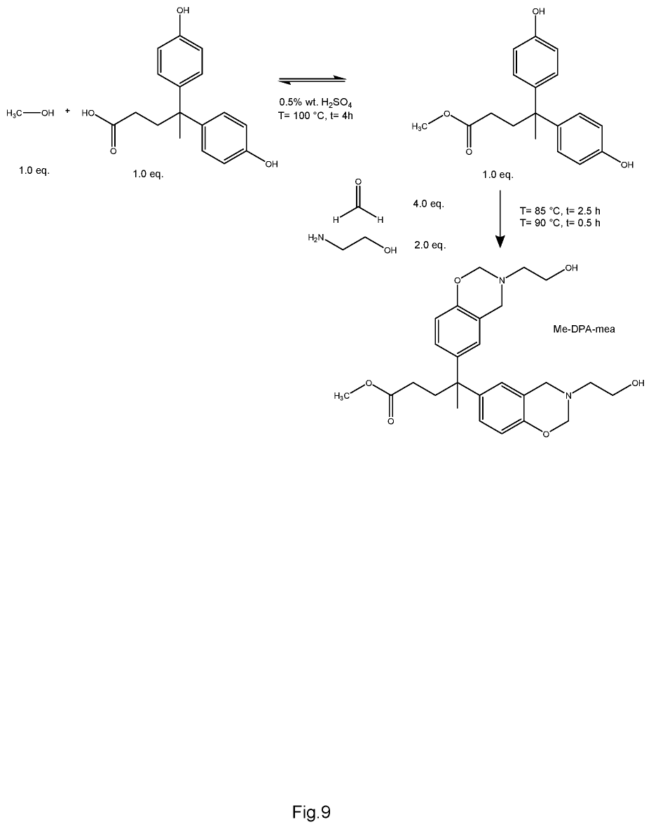

The Me-DPA-mea benzoxazine monoester containing free aliphatic hydroxyl groups was synthesized in two stages (FIG. 9). The first step, step a), corresponds to a Fischer esterification between methanol (1 eq.) and 4,4-Bis(4-hydroxyphenyl) valeric acid (DPA) (1 eq.) in presence of sulfuric acid (H₂SO₄) introduced in catalytic amount (0.5 wt %). The reactants were put together in melt at 100° C. and agitated by magnetic stirring for 14 hours, to provide methyl 4,4-bis(4-hydroxyphenyl)pentanoate (Me-DPA) (1 eq.). The second step, step b), corresponds to a Mannich condensation Me-DPA (1 eq.), mono-ethanolamine (2 eq.) and paraformaldehyde (4 eq.). All these reactants were agitated together by mechanical stirring and reacted in melt at 85° C. for 2.5 hours followed by 0.5 hours at 90° C. to provide the Me-DPA-mea monoester benzoxazine containing free aliphatic hydroxyl groups, methyl 4,4-bis(3-(2-hydroxyethyl)-3,4-dihydro-2H-benzo[e][1,3]oxazin-6-yl)pentanoate.

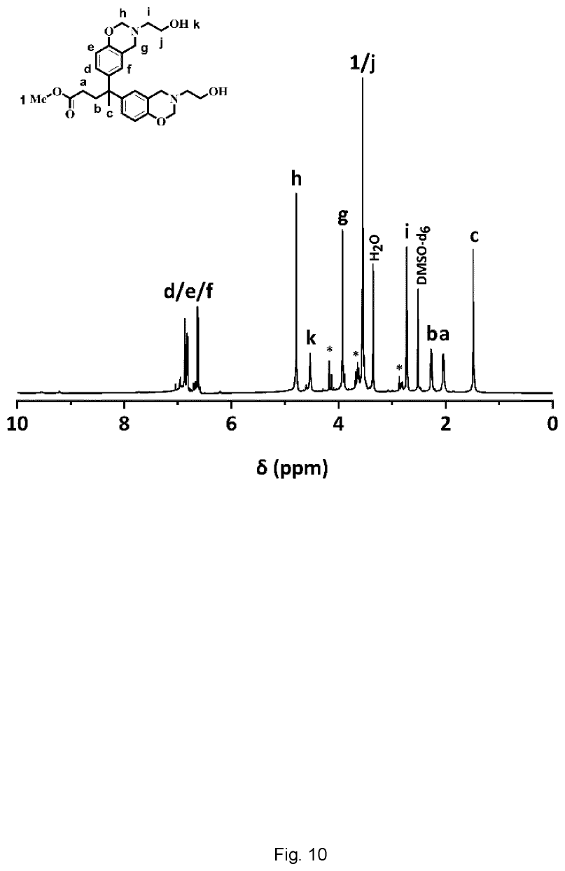

The FIG. 10 is displaying the ¹H NMR spectrum (AVANCE III HD Bruker spectrometer) of Me-DPA-mea ester-containing benzoxazine monomers.

### Example 8

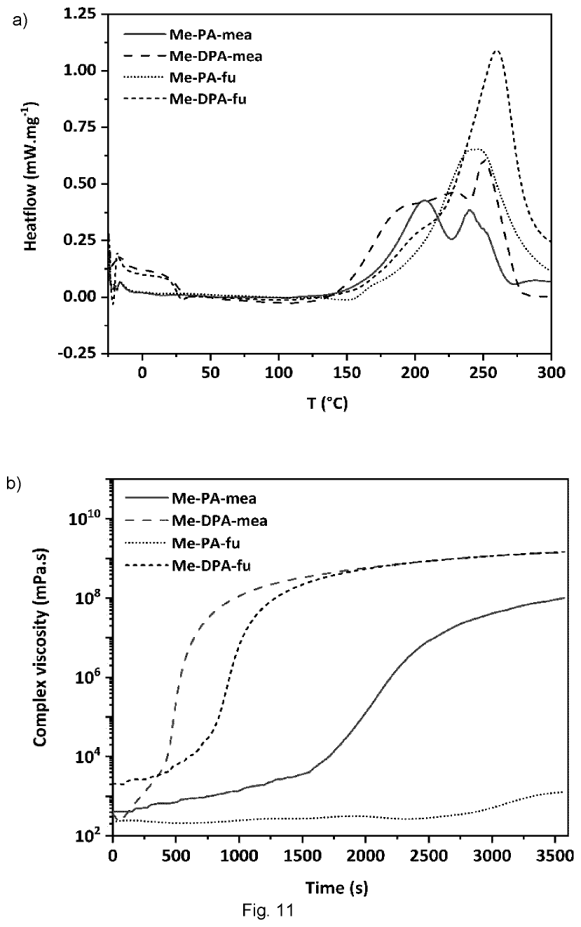

FIG. 11.a and FIG. 11.b are respectively displaying the DSC and isothermal rheology monitoring (160° C.) curves of the Me-PA-mea, Me-DPA-mea, Me-PA-fu, Me-DPA-fu (Me-PA/DPA-mea/fa) ester-containing benzoxazine monomers, “fu” designating furfurylamine. Conditions: 10° C.min⁻¹, N2 atmosphere.

Me-PA-fu: methyl 3-(3-(furan-2-ylmethyl)-3,4-dihydro-2H-benzo[e][1,3]oxazin-6-yl)propanoate;

Me-DPA-fu: methyl 4,4-bis(3-(furan-2-ylmethyl)-3,4-dihydro-2H-benzo[e][1,3]oxazin-6-yl)pentanoate;

Me-PA-fu and Me-DPA-fu ester-containing benzoxazine monomer are obtained as in Example 6 and Example 7 respectively, where furfurylamine is used instead of mono-ethanolamine.

Accordingly, FIG. 11.a is displaying the DSC thermogram of monofunctional and polyfunctional ester containing benzoxazine. The abbreviations “mea” and “fu” correspond respectively to mono-ethanolamine (terminated by free hydroxyl groups) and furfurylamine (terminated by furan groups). The DSC thermogram of furfurylamine containing benzoxazine monomers shows a first exothermic peak starting at a temperature of 150° C. This peak is associated to the ring opening of the benzoxazine rings upon heating. The ring opening of the benzoxazine rings occurred at much lower temperature in the case of I benzoxazine monomers containing ester and free alcohol groups (FIG. 11.a-Me-PA-mea and Me-DPA-mea). The first exothermic peak starts at 120° C. for a maximum located around 200° C. Transesterification reactions between ester bonds and aliphatic hydroxyl groups promote the thermal ring opening polymerization of benzoxazine monomer. The second exothermic peak corresponds to the degradation the aliphatic ester, observed in both case (mea and fu).

The curing of the Me-PA/DPA-mea/fu ester-containing benzoxazine monomers was monitored by rheological measurement in FIG. 11.b. The rheogram is performed under the following conditions: 1 Hz, with linear amplitude from 1 to 0.1%; 25 mm plates. The test is performed following a heating ramp from 80° C. to 160° C. at 15° C./min followed by an isothermal measurement at 160° C. The complex viscosity is recorded as a function of time. The term “gelation time” is defined as the time when the complex viscosity of the soften monomer increases abruptly to transform into a gel. At 160° C., the gelation time is reached after 60, 450 and 1560 s, respectively for Me-DPA-mea, Me-DPA-fu, and Me-PA-mea.

FIG. 11.a) displays DSC curves and b) displays Isothermal rheology monitoring of Me-PA/DPA-mea/fu ester-containing benzoxazine monomers.

1.-16. (canceled)

17. A benzoxazine containing free aliphatic hydroxyl groups and monoester of formula (I) 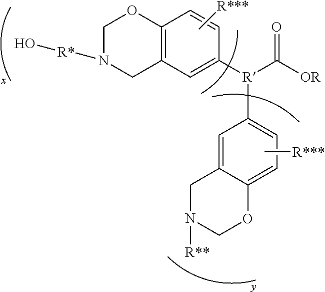
wherein
R is selected from the group consisting of a linear or branched C₁-C₁₂ alkyl or alkoxy group, a linear or branched C₂-C₆ alkenyl or alkylenoxy group, a substituted or unsubstituted linear or branched C₂-C₆ alkynyl group, a cyclo(C₃-C₆ alkyl) group, a heterocyclo(C₃-C₆ alkyl), a linear or branched C₁-C₆ alkyl or C₂-C₆ alkenyl substituted or unsubstituted phenyl group and a (CH₂)ₙ₃-phenyl group, wherein n3 is an integer from 1 to 10;
R′ is selected from the group consisting of at least one of —CH, a C—(CH₂)ₙ₃—CH₃ group, a C—(CH₂)ₙ₃—CH—(CH₃)₂ group, a C—(CH₂)ₙ₃—(CHZ)ₙ₄—(CH₃)₂ group, a C—(CH₂)ₙ₃—(CHZ)ₙ₄—(CH₂)ₙ₃—CH₃ group, a C—(CHZ)ₙ₄—(CH₂)ₙ₃—CH₃ group, a C—(CHZ)ₙ₄—[(CH₂)ₙ₃—CH₃]₂ group, a C-substituted or unsubstituted C₂-C₆ linear or branched alkenyl group, a linear or branched C₁-C₆ alkyl substituted or unsubstituted phenyl or phenyl including at least one hetero atom selected from N, O and S, a C—(CH₂)ₙ₃—C₁-C₆ linear or branched alkyl substituted or unsubstituted phenyl or phenyl including at least one heteroatom selected from N, O and S, a C—(CH₂)ₙ₃—C₁-C₆ linear or branched alkyl substituted or unsubstituted phenyl or phenyl including at least one heteroatom selected from N, O and S—(CH₂)ₙ₃—CH₃, a C—(CH₂)ₙ₃—C₁-C₆ linear or branched alkyl substituted or unsubstituted phenyl or phenyl including at least one heteroatom selected from N, O and S—(CH₂)ₙ₃—CH—(CH₃)₂, a C—(CH₂)ₙ₃—C₁-C₆ linear or branched alkyl substituted or unsubstituted phenyl or phenyl including at least one heteroatom selected from N, O and S—(CH₂)ₙ₃—(CHZ)ₙ₄—(CH₃)₂ group, a C—(CH₂)ₙ₃—C₁-C₆ linear or branched alkyl substituted or unsubstituted phenyl or phenyl including at least one heteroatom selected from N, O and S—(CHZ)ₙ₄—(CH₂)ₙ₃—CH₃ group and a C—(CH₂)ₙ₃—(CHZ)ₙ₄—C₁-C₆ linear or branched alkyl substituted or unsubstituted phenyl or phenyl including at least one heteroatom selected from N, O and S—(CH₂)ₙ₃—CH₃ group, wherein n3 and n4, independently, are an integer from 1 to 10 and Z is selected from the group consisting in a linear or branched C₁-C₆ alkyl or alkoxy group, linear or branched C₂-C₆ alkenyl or alkylenoxy group and a linear or branched C₁-C₆ alkyl or C₂-C₆ alkenyl substituted or unsubstituted phenyl group, and at least one O atom is present or not between two adjacent C, or R′ is omitted. “R′ is omitted” means that the ester moiety is directly linked to the aromatic ring, with y=0.
R* is selected from the group consisting of a linear or branched C₁-C₂₀ alkyl or alkoxy group, a cyclo(C₃-C₆ alkyl) group, a heterocyclo(C₃-C₆ alkyl) group, wherein the hetero atom is selected from N, S, and O, linear or branched C₂-C₆ alkenyl or alkylenoxy group, substituted or unsubstituted linear or branched C₂-C₆ alkynyl group, a linear or branched C₁-C₆ alkyl or C₂-C₆ alkenyl substituted or unsubstituted phenyl group, a (CH₂)ₙ₃-phenyl group and —(CH₂)ₙ₃—O—(CH₂)ₙ₄, wherein n3 and n4, independently, are an integer from 1 to 10;
R** is the same as R* and further includes a member selected from a O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group and a O-substituted or unsubstituted C₂-C₆ linear or branched alkynyl group, Z being as defined for R′, a —(CH₂)ₙ₃—C—N group, a polycyclic aromatic or heteroaromatic hydrocarbon, such as naphthalene, anthracene, fluorene, phenanthrene, optionally substituted by a linear or branched C₁-C₆ alkyl or alkoxy group, a cyclo(C₃-C₆ alkyl) group, a heterocyclo(C₃-C₆alkyl) group, a linear or branched C₂-C₆ alkenyl or alkylenoxy group, or by a substituted or unsubstituted linear or branched C₂-C₆ alkynyl group, wherein n3 and n4, independently, are an integer from 1 to 10;
R*** is selected from the group consisting in H, OH and a O-linear or branched C₁-C₆ alkyl group, and further includes a linear or branched C₁-C₁₅ alkyl group or a C₂-C₁₅ alkenyl group or 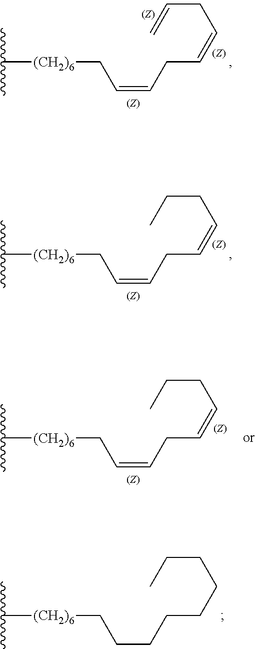
and x value is of from 0 to 1 and y value is 1-x.

18. The benzoxazine according to claim 17, wherein
R is selected from the group consisting of a linear or branched C₁-C₄ alkyl or alkoxy group, a linear or branched C₂-C₄ alkenyl or alkylenoxy group, an unsubstituted linear or branched C₂-C₄ alkynyl group, an unsubstituted phenyl group and a (CH₂)ₙ₃-phenyl group, wherein n3 is an integer from 1 to 6;
R′ is selected from the group consisting of at least one of —CH, a C—(CH₂)ₙ₃—CH₃ group, a C—(CH₂)ₙ₃—CH—(CH₃)₂ group, a C—(CH₂)ₙ₃—(CHZ)ₙ₄—(CH₃)₂ group, C—(CH₂)ₙ₃—(CHZ)ₙ₄—(CH₂)ₙ₃—CH₃ group, C—(CHZ)ₙ₄—(CH₂)ₙ₃—CH₃ group, a C—(CHZ)ₙ₄—[(CH₂)ₙ₃—CH₃]₂ group, a C-substituted or unsubstituted C₂-C₄ linear or branched alkenyl group, an unsubstituted phenyl or phenyl including at least one heteroatom selected from N, O and S, a C—(CH₂)ₙ₃-unsubstituted phenyl or phenyl including at least one heteroatom selected from N, O and S, a C—(CH₂)ₙ₃-unsubstituted phenyl or phenyl including at least one heteroatom selected from N, O and S—(CH₂)ₙ₃—CH₃, a C—(CH₂)ₙ₃-unsubstituted phenyl or phenyl including at least one heteroatom selected from N, O and S—(CH₂)ₙ₃—CH—(CH₃)₂, a C—(CH₂)ₙ₃-unsubstituted phenyl or phenyl including at least one heteroatom selected from N, O and S—(CH₂)ₙ₃—(CHZ)ₙ₄—(CH₃)₂ group, a C—(CH₂)ₙ₃-unsubstituted phenyl or phenyl including at least one heteroatom selected from N, O and S—(CHZ)ₙ₄—(CH₂)ₙ₃—CH₃ group and a C—(CH₂)ₙ₃—(CHZ)ₙ₄— unsubstituted phenyl or phenyl including at least one heteroatom selected from N, O and S—(CH₂)ₙ₃—CH₃ group, wherein n3 and n4, independently, are an integer from 1 to 6 and Z is selected from the group consisting in a linear or branched C₁-C₄ alkyl or alkoxy group, linear or branched C₂-C₄ alkenyl or alkylenoxy group and an unsubstituted phenyl group, and at least one O atom is present or not between two adjacent C;
R* is selected from the group consisting of a linear or branched C₁-C₆ alkyl or alkoxy group, a linear or branched C₂-C₄ alkenyl or alkylenoxy group, an unsubstituted linear or branched C₂-C₄ alkynyl group, an unsubstituted phenyl group, a (CH₂)ₙ₃-phenyl group and —(CH₂)ₙ₃—O—(CH₂)ₙ₄, wherein n3 and n4, independently, are an integer from 1 to 6;
R** is the same as R* and further includes a member selected from a O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group and O-substituted or unsubstituted C₂-C₄ linear or branched alkynyl group, Z being as defined above, a —(CH₂)ₙ₃—C—N group, a cyclo(C₃-C₄ alkyl), a heterocyclo(C₃-C₄alkyl) group, a polycyclic aromatic or heteroaromatic hydrocarbon (PAH), wherein the hetero atom is selection from N, S, and O, such as naphthalene, anthracene, fluorene, furane, optionally substituted by a linear or branched C₁-C₄ alkyl or alkoxy group, a linear or branched C₂-C₄ alkenyl or alkylenoxy group, or by a substituted or unsubstituted linear or branched C₂-C₄ alkynyl group, wherein n3 and n4, independently, are an integer from 1 to 6;
R*** is selected from the group consisting in H, OH and a O-linear or branched C₁-C₄ alkyl group, and further includes a linear or branched C₁-C₁ alkyl group or C₂-C₁₀ alkenyl group or

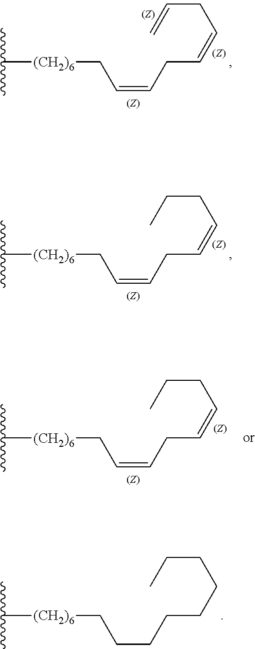

19. The benzoxazine according to claim 17, wherein
R is selected from the group consisting of groups —CH₃, —(CH₂)ₙ₃—CH₃, —(CH₂)ₙ₃—CH—[(CH₂)ₙ₃—CH₃]₂, —C(CH₃)₃, —(CH₂)ₙ₃—(C₆H₅), —(CH₂)ₙ₃—CH═CH₂ and —(CH₂)ₙ₃—C═CH, wherein n3 is an integer from 1 to 5;
R′ is selected form the group consisting of groups —CH, —C—(CH₂)₂—C(CH₃), —C—CH(CH₂CH₃), —C(CH₂CH₂CH₃), —C—CH₂(CH₂)₃CH₃, —C—CH₂(CH₂)₄CH₃, —C(C₆H₅), —C(CH₃)CH₂, C(CH₃)CH₂CH₂ and —C(C₆H₅)CH₂—CH₃;
R* is selected from the group consisting of groups —CH₃, —(CH₂)ₙ₃—CH₃, —(CH₂)ₙ₃—CH—[(CH₂)ₙ₄—CH₃]₂, —C(CH₃)₃, (CH₂)ₙ₃—(C₆H₅), —(CH₂)ₙ₃—CH═CH₂, —(CH₂)ₙ₃—C═CH, —(CH₂)ₙ₃—O—(CH₂)ₙ₄ wherein n3 and n4 independently are integer from 1 to 4, phenyl, and —(CH₂)₃-phenyl.
R** is selected from the group consisting of groups CH₃, —(CH₂)ₙ₃—CH₃, —(CH₂)ₙ₃—CH—[(CH₂)ₙ₄—CH₃]₂, —C(CH₃)₃, (CH₂)ₙ₃—(C₆H₅), —(CH₂)ₙ₃—CH═CH₂, —(CH₂)ₙ₃—C═CH, 0-(CH₂)ₙ₃—C═CH, O—(CH₂)ₙ₃—C═N, (CH₂)ₙ₃—C═N, and —(CH₂)ₙ₃-substituted or unsubstituted furan, phenyl, and wherein n3 and n4 independently are integer from 1 to 4;
R*** is selected from the group consisting in H, OH and O-linear or branched C₁-C₃ alkyl group, and further includes linear or branched C₁-C₆ alkyl group or C₂-C₆ alkenyl group or 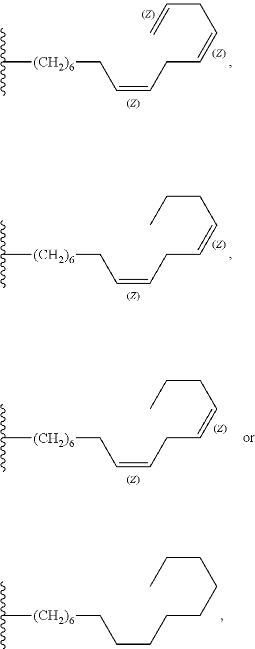
H being the most preferred.

20. A process for producing a benzoxazine containing free aliphatic hydroxyl groups and monoester of formula (I) comprising the following steps of:
a) reaction of a phenolic acid derivative of formula (II), comprising at least one R*** group, 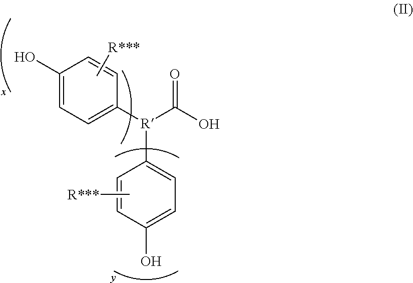
with a monofunctional oligomer or molecule of formula (III)
R—OH (III)
at a temperature of from 80° C. to 200° C., during 12 h-48 h, in a presence of a Bronsted type acid catalyst, resulting in a phenol terminated oligomer or molecule of formula (IV) 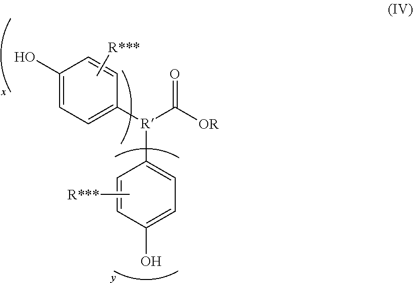
and
b) reaction of the phenol terminated oligomer or molecule of formula (IV) with a mixture of
an amino-alcohol of formula (V): 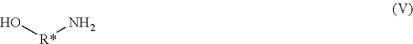
a primary amine derivative of formula (VI)
R**—NH₂ (VI), and
paraformaldehyde of formula (VII) 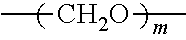
m=8-100
at a temperature range of from 80° C. to 100° C., from 1 h to 48 h, under stirring, wherein R, R′, R*, R**, R***, x and y are, independently, as defined in claim 18, with the proviso that when the at least one R*** of the phenolic acid derivative is in ortho position with regard to —OH group, then R*** is H.

21. The process according to claim 20, wherein the phenolic acid derivative (formula (II)) includes from 1 to 4 R*** group(s), related to the substitution of the phenolic ring.

22. The process according to claim 20, wherein the phenolic acid derivative is selected from the group consisting of mono-, di-, tri-hydroxybenzoic acid derivatives, anacardic acid derivatives, hydroxycinnamic acid derivatives, aliphatic X-hydroxyphenyl acid derivatives, wherein X is 2-4, and aliphatic diphenolic acid derivatives, or mixtures thereof.

23. The process according to claim 20, wherein the respective stoichiometry of starting reactants on step a), phenolic acid derivative:monofunctional molecule or oligomer is 1.0-3.0 eq.:1.0 eq, resulting in an 1.0 eq. of phenol terminated oligomer or molecule.

24. The process according to claim 20, wherein the primary amine derivatives bear R** having the definition of R* and are further selected from the group consisting in allylamine, methylamine, ethylamine, propylamine, butylamine, isopropylamine, hexylamine, cyclohexylamine, stearylamine, 2-aminofluorene, aminophenyl acetylene, propargyl ether aniline, 4-aminobenzonitrile, furfurylamine and aniline, or mixtures thereof.

25. The process according to claim 20, wherein the amino-alcohol of formula (V) is selected from the group consisting of 2-aminoethanol, 2-amino-2-methylpropanol, 5-aminopentan-1-ol, heptaminol and diglycolamine, or mixtures thereof.

26. The process according to claim 20, wherein the respective stoichiometry of starting reactants on step b), phenol terminated oligomer or molecule:amino-alcohol:primary amine derivative:paraformaldehyde is 1.0 eq.:x(1.0 eq-18.0 eq): y(1.0 eq-18.0 eq):2.0-36.0 eq, resulting in an 1.0 eq. of the benzoxazine containing free aliphatic hydroxyl groups and monoester, wherein x=0-1, and y=1-x.

27. A benzoxazine ring-opening polymerisation (ROP) catalyst comprising a benzoxazine containing free aliphatic hydroxyl groups and monoester of formula (I) 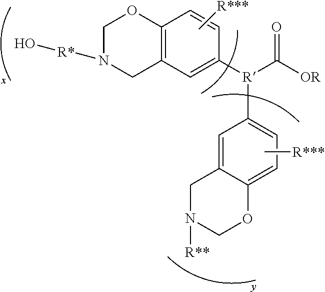
wherein
R is selected from the group consisting of a linear or branched C₁-C₁₂ alkyl or alkoxy group, a linear or branched C₂-C₆ alkenyl or alkylenoxy group, a substituted or unsubstituted linear or branched C₂-C₆ alkynyl group, a cyclo(C₃-C₆ alkyl) group, a heterocyclo(C₃-C₆ alkyl), a linear or branched C₁-C₆ alkyl or C₂-C₆ alkenyl substituted or unsubstituted phenyl group and a (CH₂)ₙ₃-phenyl group, wherein n3 is an integer from 1 to 10;
R′ is selected from the group consisting of at least one of —CH, a C—(CH₂)ₙ₃—CH₃ group, a C—(CH₂)ₙ₃—CH—(CH₃)₂ group, a C—(CH₂)ₙ₃—(CHZ)ₙ₄—(CH₃)₂ group, a C—(CH₂)ₙ₃—(CHZ)ₙ₄—(CH₂)ₙ₃—CH₃ group, a C—(CHZ)ₙ₄—(CH₂)ₙ₃—CH₃ group, a C—(CHZ)ₙ₄—[(CH₂)ₙ₃—CH₃]₂ group, a C-substituted or unsubstituted C₂-C₆ linear or branched alkenyl group, a linear or branched C₁-C₆ alkyl substituted or unsubstituted phenyl or phenyl including at least one hetero atom selected from N, O and S, a C—(CH₂)ₙ₃—C₁-C₆ linear or branched alkyl substituted or unsubstituted phenyl or phenyl including at least one heteroatom selected from N, O and S, a C—(CH₂)ₙ₃—C₁-C₆ linear or branched alkyl substituted or unsubstituted phenyl or phenyl including at least one heteroatom selected from N, O and S—(CH₂)ₙ₃—CH₃, a C—(CH₂)ₙ₃—C₁-C₆ linear or branched alkyl substituted or unsubstituted phenyl or phenyl including at least one heteroatom selected from N, O and S—(CH₂)ₙ₃—CH—(CH₃)₂, a C—(CH₂)ₙ₃—C₁-C₆ linear or branched alkyl substituted or unsubstituted phenyl or phenyl including at least one heteroatom selected from N, O and S—(CH₂)ₙ₃—(CHZ)ₙ₄—(CH₃)₂ group, a C—(CH₂)ₙ₃—C₁-C₆ linear or branched alkyl substituted or unsubstituted phenyl or phenyl including at least one heteroatom selected from N, O and S—(CHZ)ₙ₄—(CH₂)ₙ₃—CH₃ group and a C—(CH₂)ₙ₃—(CHZ)ₙ₄—C₁-C₆ linear or branched alkyl substituted or unsubstituted phenyl or phenyl including at least one heteroatom selected from N, O and S—(CH₂)ₙ₃—CH₃ group, wherein n3 and n4, independently, are an integer from 1 to 10 and Z is selected from the group consisting in a linear or branched C₁-C₆ alkyl or alkoxy group, linear or branched C₂-C₆ alkenyl or alkylenoxy group and a linear or branched C₁-C₆ alkyl or C₂-C₆ alkenyl substituted or unsubstituted phenyl group, and at least one O atom is present or not between two adjacent C, or R′ is omitted. “R′ is omitted” means that the ester moiety is directly linked to the aromatic ring, with y=0.
R* is selected from the group consisting of a linear or branched C₁-C₂₀ alkyl or alkoxy group, a cyclo(C₃-C₆ alkyl) group, a heterocyclo(C₃-C₆ alkyl) group, wherein the hetero atom is selected from N, S, and O, linear or branched C₂-C₆ alkenyl or alkylenoxy group, substituted or unsubstituted linear or branched C₂-C₆ alkynyl group, a linear or branched C₁-C₆ alkyl or C₂-C₆ alkenyl substituted or unsubstituted phenyl group, a (CH₂)ₙ₃-phenyl group and —(CH₂)ₙ₃—O—(CH₂)ₙ₄, wherein n3 and n4, independently, are an integer from 1 to 10;
R** is the same as R* and further includes a member selected from a O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group and a O-substituted or unsubstituted C₂-C₆ linear or branched alkynyl group, Z being as defined for R′, a —(CH₂)ₙ₃—C—N group, a polycyclic aromatic or heteroaromatic hydrocarbon, such as naphthalene, anthracene, fluorene, phenanthrene, optionally substituted by a linear or branched C₁-C₆ alkyl or alkoxy group, a cyclo(C₃-C₆ alkyl) group, a heterocyclo(C₃-C₆alkyl) group, a linear or branched C₂-C₆ alkenyl or alkylenoxy group, or by a substituted or unsubstituted linear or branched C₂-C₆ alkynyl group, wherein n3 and n4, independently, are an integer from 1 to 10;
R*** is selected from the group consisting in H, OH and a O-linear or branched C₁-C₆ alkyl group, and further includes a linear or branched C₁-C₁₅ alkyl group or a C₂-C₁₅ alkenyl group or 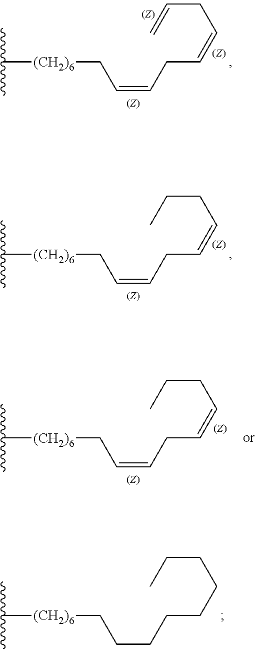
and x value is of from 0 to 1 and y value is 1−x.
